(19) United States
(12) Patent Application Publication
(10) Pub. No.: US 2007/0253942 A1
(43) Pub. Date: Nov. 1, 2007

(54) Lectin-directed prodrug delivery system

(75) Inventor: Benjamin Guy Davis, Oxford (GB)

Correspondence Address: NIXON & VANDERHYE, PC, 901 NORTH GLEBE ROAD, 11TH FLOOR, ARLINGTON, VA, 22203, US

(73) Assignee: Isis Innovation Limited, Oxford (GB)

(21) Appl. No.: 11/785,261

(22) Filed: Apr. 16, 2007

Related U.S. Application Data
(63) Continuation of application No. 10/473,814, filed on Mar. 9, 2004, now abandoned.

(30) Foreign Application Priority Data
Apr. 3, 2001 (GB) 0108332.8

(51) Int. Cl. A61K 38/46; A61P 43/00; C12N 9/14; C12P 19/00

(52) U.S. Cl. 424/94.3; 435/195; 435/72

(57) ABSTRACT

The invention provides a kit for lectin-directed prodrug delivery comprising a prodrug and a lectin-directed glycoconjugate, wherein the glycoconjugate is adapted to cleave the prodrug and thereby release the drug. The glycoconjugate generally comprises an enzyme conjugated to a carbohydrate moiety which binds to a lectin. The invention also provides novel glycoconjugates and novel methods of synthesizing prodrugs.

### FIELD OF THE INVENTION

The present invention relates to the targeted delivery of prodrugs to cells.

### BACKGROUND TO THE INVENTION

Increasingly, carbohydrates are being investigated as the key factors in recognition processes, particularly in cell-cell recognition.ⁱ Carbohydrates are often found as conjugates of other biomolecules, forming glycolipids and glycoproteins. The carbohydrate moiety brings its own particular functionality to these structures, increasing hydrophilicity or enhancing conformational stability.ⁱⁱ

Naturally occurring glycoconjugates exist as glycoforms, with slight variations in structure from one glycoform to another,ⁱⁱⁱ resulting in slight changes in physical properties. This does however lead to the desire to produce homogeneous glycoconjugates and so a number of strategies towards this goal have been developed and are known in the art, as illustrated in Table 1. Many of these techniques suffer from lack of specificity and control of the extent of glycosylation depends on the length of time and concentration allowed for glycosylation to occur, ie the prevalence of a particular amino acid residue and the kinetics of the reaction between residue and glycosylating agent.

An improved approach gives site-directed mutagenesis and chemical modification by introducing the single free cysteine residue of bovine serum albumin (BSA) at specific sites within the peptide chain and glycosylation with cysteine selective reagents (Table 1, Entry 6).

TABLE 1
Synthetic glycosylation techniques

| | | Amino | | Time and | |
|---|---|---|---|---|---|
| | | acid | | extent of | |
| Name | Scheme | residue | pH | modification | Solvent |
| Reductive amination | 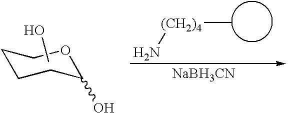 | Lysine | 7 | 4 of 59 lys of BSA in 2 weeks | Aqueous |
| | 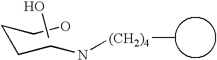 | | | | |
| IME thioglycosides | 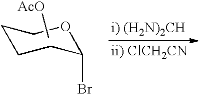 | Lysine | 8.5 | 41 of 59 lys of BSA in 2 hours | Aqueous sodium borate buffer |
| | 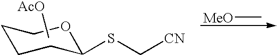 | | | | |
| | 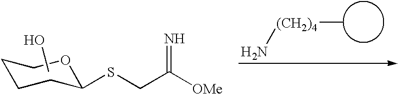 | | | | |
| | 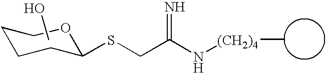 | | | | |
| N-acetyl glucosamines | 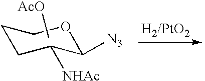 | Cysteine | | 1 of 1 cys in BSA in 40 minutes | Aqueous ammonium carbonate |
| | 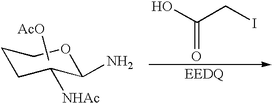 | | | | |
| | 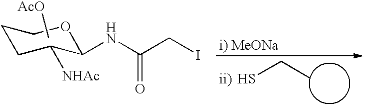 | | | | |
| | 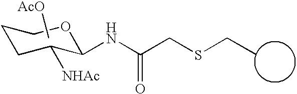 | | | | |
| 5-nitropyridine- 2-sulfenyl activated^{iv} | 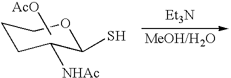 | Cysteine | 5 | 1 of 1 cys of BSA in 2-15 minutes | Aqueous ammonium acetate buffer |
| | 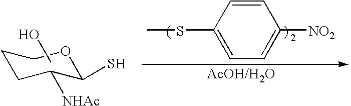 | | | | |
| | 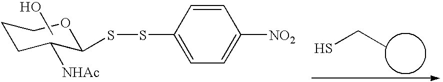 | | | | |
| | 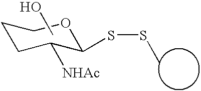 | | | | |
| Diethyl squarate | 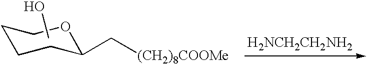 | Lysine | 9 | 13 of 59 lys of BSA in 16 hours | Aqueous KHCO₃/ Na₂B₄O₇buffer |
| | 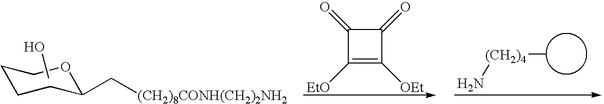 | | | | |
| | 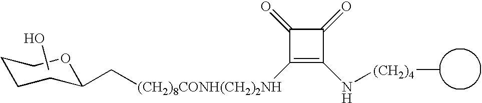 | | | | |
| Methanethio sulfonates | 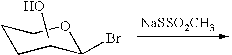 | Cysteine | 5.5- 9.5 | 1 of 1 mutated cys in SBL | Aqueous buffer |

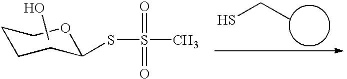

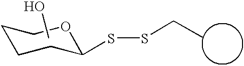

100% delivery of any drug to its intended site of action is currently impossible. The natural metabolism of compounds, natural and foreign, in the body ensures that the drug compound is converted to a form that the body is able to excrete with greater ease. Even if a drug is designed to withstand in vivo metabolism it is still essential that the drug reaches the desired target and therefore that some direction or targeting is built into the drug.

There is a need to reduce the dose administered and to cut dosage so as to prevent side effects traditionally found when dose is increased to overcome loss of the drug in vivo. The blood distributes a drug around the body. Transfer from the blood to other sites of activity—the intended site of action and of elimination—is generally by diffusion and is therefore controlled by the concentration gradient between blood plasma and target cells, differential delivery of a drug to the site of action and the selectivity imposed by receptors which may or may not accept the drug. Large pores in the capillary endothelia allows rapid transfer to the interstitial fluid, and thereby to the cell surface; Transfer across the lipid cell membrane is now dependant on the hydrophilic or lipophilic properties of the drug.

It is rare that the specific properties of a molecule required to bring about some response in vivo are also suited to the specific delivery of that molecule to the desired site in the body and crossing a lipophilic membrane.

A technique to increase the selectivity of a drug, by enhancing the ratio between the activity and toxicity of a drug uses prodrugs, or Active Drug Production. Prodrugs generally consist of two moieties, one to allow uptake or to mask the toxic properties of the drug part or to protect the drug and the other to bring about some effect, which are cleaved near the site of action in preferably in quantitative yield. Other advantages of prodrug therapy include reduction of gastrointestinal irritability and pain at injection site, improvement of taste and odour, increased chemical stability.

Enzymes found naturally in the body may be used to carry out this cleavage. One group of strategies involves “bipartate” drug delivery, where two different moieties are independently administered. The first, normally an enzyme, specifically targets a cell type and provides a mechanism for the activation of the second, normally a prodrug. However this has been limited to specific therapies.

ADEPT (antibody-directed enzyme prodrug therapy also known as Antibody Directed Catalysis—ADC) involves the introduction of enzyme-antibody conjugates which are complementary to surface antigens of a target cell, followed by a prodrug which is selectively cleaved by the enzyme previously introduced.

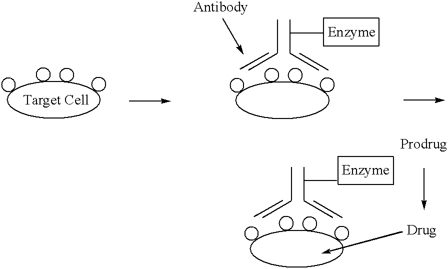

Antigens are one of the few physical characteristics that differ between cells and so it is possible to target tumour cells in this way. The elegance of ADEPT is countered by the problems of controlling such a complex system. Determining the antibody-enzyme conjugate to be used does of course depend on the antigen to be targeted and the prodrug to be delivered, but more specifically it is essential that control over the pharmacokinetics of the conjugate be controlled. The conjugate must be localised at the tumour cells before administration of the prodrug, so as to allow release of the active drug from the parent only at the desired site. Careful modification of the antibody of choice and the gross molecular weight of the conjugate does bestow some control. The antibody should have high affinity only to the tumour cell antigen, and its covalent attachment to the enzyme should not affect either the antibody or enzyme function.^{v}

Mammalian or non-mammalian enzyme is employed, non-mammalian reducing the risk that any enzyme present in vivo would cleave the parent prodrug, so allowing even greater control over drug concentration at the target cells. But problems arise due to the immunogenicity of the foreign antibody-enzyme conjugate.^{vi} This can be circumvented by the use of mammalian enzymes, but exclusive production of active drug at the target site is unlikely.^{vii} A number of techniques^{viii} have been developed to prevent immunogenicity caused by the presence of foreign enzymes and antibodies, including the use of humanised antibodies and a complete redesign of the antibody in question.

Current methodologies for the directed targeting of prodrugs are variously over-complicated, highly situation dependent, and expensive. There is therefore a need for a new targeted prodrug system.

The presence of lectins on the surface of cells is widely accepted and exploited. Lectins are carbohydrate-binding proteins in the form of multivalent ligands with binding sites that recognise a particular sugar residue or sequence of sugar residues. The fact that oligosaccharides are responsible for much intracellular signalling, bringing about a physiological response, is also commonplace.^{ix} Lectins were first discovered in 1888, and since then their investigation has lead to a plethora of articles and reviews.^{x} Known is mannose directed uptake of a beneficial enzyme at a specific mannose-binding lectin on a cell (macrophage) surface.

### SUMMARY OF THE INVENTION

We have now surprisingly found that by a novel use of separate glycoproteins and prodrug conjugates, we can provide a protein or enzyme which is directed to a particular target, a desired cell type having a specific lectin in its membrane, by the exploitation of the specific lectin-carbohydrate interaction, and we can provide a prodrug which is directed to the enzyme, by the exploitation of enzyme affinity, and which is processed to release drug. The system is illustrated in the scheme for the enzyme naringinase.

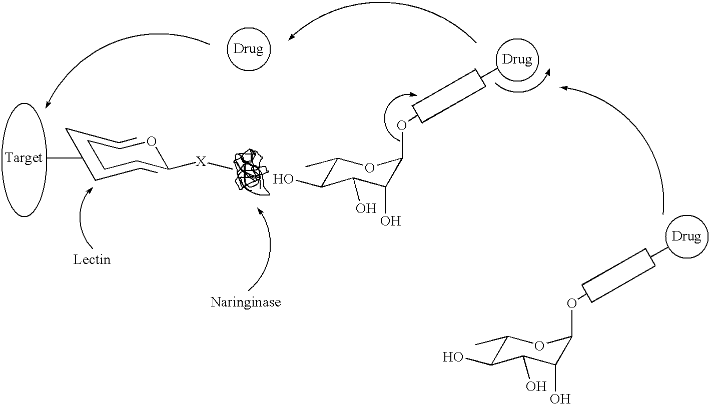

In the broadest aspect of the invention there is provided a kit for lectin-directed prodrug delivery comprising a lectin-directed glycoconjugate and a corresponding prodrug wherein the glycoconjugate is adapted to cleave the prodrug, with release of drug.

### DETAILED DESCRIPTION OF THE INVENTION

Reference herein to a glycoconjugate, more specifically a glycoprotein or glycoenzyme, is to a carbohydrate, preferably an (oligo)saccharide, also referred to herein as a sugar, which in turn is conjugated to a molecule, more specifically a protein or an enzyme capable of bringing about a specific chemical reaction. Glycosylation confers recognition as well as affecting function and transport of the protein or enzyme.

Reference herein to a prodrug is to a pharmaceutically useful molecule, hereinafter a drug, which includes a deactivating or masking group, hereinafter a cap, which renders the molecule pharmaceutically inactive, and which is capable of reaction with the glycoconjugate to remove the cap, releasing the pharmaceutically active drug.

According to the present invention therefore we have combined numerous separate, but synergic, mechanisms into one elegant lectin-enzyme activated prodrug system (LEAP) combining lectin binding for the specific targeted delivery of an enzyme to a cell type, with the controlled release of a prodrug by that enzyme which allows control over the whole delivery process

This “building block” system allows the potential construction of a diverse array of combinations of enzyme, enzyme glycosylation and prodrug, potentially allowing the selected targeting of any drug to any cell type with suitable lectins.

In one embodiment the kit according to the invention comprises one lectin-directed glycoconjugate and at more than one corresponding prodrug. In an alternative embodiment the kit according to the invention comprises more than one lectin-directed glycoconjugate and one corresponding prodrug.

This provides an additional pick and mix type methodology for the kit adapted for the treatment of a specific condition with a range of drugs, or conversely for the treatment of a variety of conditions with a single multieffect drug, and allows a clinician to select and tailor a methodology and kit to target different tissue with the same activity or the same tissue with different activities. This can be useful for a specific individual, but also importantly to speed up treatment regimes, avoid conflict of treatments and the like in the case of a complex conditions or sensitive patients.

The site and nature of glycosylation determines the effect imparted on the protein or enzyme and glycosylation affecting enzymatic activity may be near to or distant from the active site and may be of natural or synthetic type. Glycosylation may be site specific and is of the N-glycan or O-glycan naturally occurring type, forming an aspartylglycosylamine linkage beta to a serine or threonine residue, or is an N-acetyl or xylose linkage to serine or threonine. Synthetic type glycosylation is usually to the lysine residue but is more preferably to a cysteine residue. Glycosylation may at a single or plural sites in any given protein, and is controlled by kinetics.

There is provided a method for the selection of a kit as hereinbefore defined, comprising determining the lectin type or cell type or location to target for a given purpose and the drug type to be administered, selecting a carbohydrate recognised by and binding to the selected lectin type or cell type or location, selecting a suitable cap to form a prodrug, selecting a pharmaceutically acceptable molecule (protein or enzyme) to conjugate therewith and which includes in its repertoire of reaction substrates a group which is suitable as a cap as hereinbefore defined. Accordingly selection of carbohydrate is dependent on lectin recognition for the desired target cell type and selection of molecule for glycoconjugate is dependent on reaction and release of the capping moiety of the desired drug which it is desired to deliver. Conveniently the cap may comprise the corresponding carbohydrate known to be hydrolysed by a chosen enzyme.

A glycoconjugate as hereinbefore defined is selectively glycosylated to comprise any desired carbohydrate recognised by a specific lectin to provide specific targeting to a desired cell type comprising the lectin.

The carbohydrate moiety is selected from specific oligosaccharides to be specific to different lectins, and thereby to various cell types, as hereinbefore defined.

Cell-oligosaccharide combinations may be selected from for example:

Hepatocytes—galactose specific lectin^{xi}

Macrophages—mannose specific lectin^{xii}

Macrophages—mannose and mannose-6-phosphate specific lectin^{xiii}

Monocytes—mannose-6-phosphate lectin^{xiv}

Glycosyl groups may be linked directly or via a linker moiety, or a glycosylating precursor may comprise a linker moiety in addition to the carbohydrate moiety.

A linker moiety may be any suitable group which is useful in glycosylation, and is preferably selected from the following groups in which —(c)- indicates the linkage to the carbohydrate, (c)- indicates a carbohydrate moiety, —(e)—indicates the link to an atom or group X in a nucleophilic group in the molecule to be glycosylated, for example each X is independently selected from NH, S or O in a nucleophilic group in an enzyme, and -(e) indicates the molecule to be glycosylated, for example an enzyme; each Y independently is selected from NH, S or O in the linker and each Z is independently selected from NH, S, CH2 or O in a carbohydrate; and each n, a, b, c, d, e independently is selected from 1 to 10 or any subrange thereof for example 1 to 5 or 1 to 3:

(a) an imino alkyl group of the general formula Ia
-(c)-SCH2C(NH)-(e)- Ia
preferably of the general formula Ia′
[-(c)-XCH2COX(CH2)n]2N(CH2)nNHCO(CH2)nSCH2C(NH)-(e)- Ia′
wherein X is N and Z is as hereinbefore defined;
the linker of Formula Ia or Ia′ is suitably obtained by the known IME reaction as hereinbefore defined for example the corresponding imino 2-methoxy ethyl thio precursor will react with the side chain N of lysine to give the linker group —CH2C(NH)NH-(e);
the imino 2-methoxy ethyl thio precursor to Formula Ia or Formula Ia′ is suitably obtained by the known methoxide transformation of the corresponding cyano thioamide, which is obtained (Ia) by known means or (Ia′) by the novel reaction Scheme Ia′:
   [(c)-XCH2COX(CH2)n]v′N(H)v(CH2)nNH2+SC(O)(CH2)3+L(CH2)nCN→[(c)-XCH2COX(CH2)n]v′2N(H)v(CH2)nNHCO(CH2)nSCH2CN
   wherein SC(O)(CH2)₃ is the cyclic gamma-thiobutyrolactone and L is a leaving group;

(b) a direct link of formula Ib
-(c)-(e)- Ib
Wherein each of X and Z are S;
the group Ib is suitably obtained by known means via the 5-nitro pyridine-2-sulfenyl route or the methane thio sulfonate route as hereinbefore described

(c) a group of the formula Ic
-(c)-(CH2)nC(O)-(e)-
or -(c)-C(O)(CH2)n-(e) Ic
wherein Z is N and X is S;
the group Ic is suitably obtained by known means via the N-acetylglucosamine route as hereinbefore described

(d) a group of formula Id
-(c)-CONH(CH2)nNH—C(C(O))═C(C(O))-(e)- Id
Wherein Z is CH2, X is NH and —C(C(O))═C(C(O))— is the 4-membered cyclic squarate;
the group Id is suitably obtained by known means via the squarate route as hereinbefore described.

Preferably the carbohydrate moiety is present in suitable amount in the range 1 to 400 sugars, for example 1 to 20 sugars per site, and in 1 to 20 sites per glycoconjugate. Glycosylation may be in the amount 1 to 100% of possible sites for a given conjugate molecule, for example 1 to 45% or 1 to 60% possible sites for a given enzyme molecule.

The carbohydrate moiety of the glycoconjugate may be any mono, di or polysaccharide, which may be linear or branched, and may comprise additional functional groups and comprise one or a combination of the known saccharide subunits selected from, for example, the classes of mannose, galactose, glucose, fucose, N-acetylglucosamine, rhamnose, saccharides and glycoconjugates thereof, such as L-rhamnose (1-OH, 2-OH, 3-OH, 4-OH, 5-Me), L-deoxyrhamnojurimycin (1-, 2-OH, 3-OH, 4-OH, 5-Me) and other known members of the saccharide classes.

Preferably the glycosyl unit and linker in combination provides a single or plural binding sites for targetting lectin, ie comprises a mono, di, poly or dendrimeric glycosyl group. As lectin is itself a ligand it favours binding of multidentate ligands, preferably the glycosyl unit and linker comprises a divalent dendrimeric system of general formula II 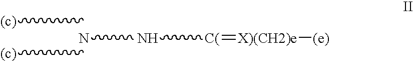
wherein [symbol] indicates a hydrocarbyl moiety, preferably of general formula
[[(c)-C(═X)Y(CH₂)n]v′N(H)v(CH₂)n]v″N(H)ᵥCO(CH₂)nY(CH₂)nC(═X)(CH₂)n-(e) II
wherein the linkage (c)- and -(e) is via respectively group Z or X and is selected from O, S, CH2 or NH as hereinbefore defined, n is as hereinbefore defined, the sum of v and v′ and of v and v″ independently equal the valence of N, wherein v is 0 or 1 and v′ and v″ are independently 1 or 2 representing mono or polyvalent, eg di, tri or tetravalent glycan constructed from repeating branching subunits, hexa or octavalent glycan may be constructed from repeating branching subunits in corresponding manner.

The enzyme may be selected from a range of enzymes of the glycosidase, hydrolase or lyase classes, allowing a wide spectrum of prodrugs to be investigated. Enzymes are commercially available (Sigma) or may be obtained by known means, suitably from culture or the like. Suitably the enzyme is selected from any non-mammalian enzymes having no mammalian equivalent nor being a variant of a mammalian equivalent whereby the enzyme introduces a novel catalytic activity to the organism, this activity being the activity required for drug release from prodrug thereby ensuring that the system will not target any undesired locations.

Enzymes with mammalian equivalent or mammalian enzymes may be considered, with suitable modification or inhibition of the natural mammalian function.

Non-mammalian enzymes include the rhamnopyranosidases, beta-lactamases, penicillin V amidases; enzymes with mammalian equivalent include beta-glucuronidases; mammalian enzymes include alpha-galactosidases and alkaline phosphatases.

Preferably an enzyme comprises an enzyme of the class of rhamnopyranosidases since this is not found naturally in mammals, more preferably α-L-rhamnopyranosidases, most preferably comprises naringinase, alpha-L-rhamnosidase, hesperidinase or an analogue or amino-terminal sequence thereof.

More preferably the enzyme comprises naringinase, an analogue or amino-terminal sequence thereof, for example selected from wild type naringinase (N-WT), deglycosylated naringinase (N-DG) and otherwise functionalised naringinase (N-F) to confer properties of heat stability, activity, selectivity, to ease in formulation by solubility, solvent resistance, dispersion, to enhance taste, odour and the like; or comprises other enzymes such as lactamases, proteases, glycosidases, nitrilases, esterases, lipases, amidases, aldolases, hydrolases, lyases and the like.

Different cultures of enzyme may give different prodrug release specificity, for example enzymes may be commercially available (Sigma) or prepared by known means for example by fermenting in Penicillum sp. (deposited as DSM 6825, DSM 6826 at the Deutschen Sammlung von Mikroorganismen und Zellkulturen GmbH), separating off the biomass from the culture broth and concentrating the culture supernatant, as disclosed in U.S. Pat. No. 5,468,625. This enzyme has been isolated and has sequence including the active N-terminal sequence

Naringinase: (96000D) A S V P X G E X I L A P S SI E L I P T

Alpha-L-rhamnosidase 6826, peptide I (96000D)

D-T-N-D-Q-T-S-A-K-V-D-R-G-T-F-D-D-P-A-

And Alpha-L-rhamnosidase 6826, peptide II (93500D)

F-F-G-S-X1 (cysteine?)-Q-S-L-Y-L-K-L-V-L-K-F-G-T-L-B-D (X2?)A

Alpha-L-rhamnosidases (EC number 3.2.1.40) are found in a number of organisms sometimes with slightly different activities and hydrolyse the terminal non-reducing alpha-L-rhamnose residues in alpha-L-rhamnosides.

A separate study^{xv} has isolated an α-L-rhamnopyranosidase from Aspergillus aculeatus that can hydrolyse rhamnose from the non-reducing end of the rhamnogalacturonan fragments of pectin. Nevertheless studies have shown that of the different methyl rhamnopyranosides, alpha-L-rhamnopyranosidase is specific to methyl alpha-L-rhamnopyranoside.

Preferably the enzyme is also selected according to its ability to both synthesise and hydrolyse the glycosyl linkage of a desired drug. For example a β-galactosidase from Bacillus circulans has been shown to catalyse the formation (and therefore may be expected to catalyse its hydrolysis) of ethyl 1-thio-(β-D-galactopyranosyl)-O-β-D-glycopyranosyl disaccharides,^{xvi} employing a range of 1-thio-β-D-glycopyranosides as glycosyl acceptors and p-nitrophenyl β-D-galactopyranoside as glycosyl donor (Representation 1). Yields in the range 10-60% are commonly accepted with glycosidase transformations, as was found with this example. In the same way rhamnosidases can be used to make and break rhamnoside bonds.
Representation 1: Enzymatic Disaccharide Synthesis

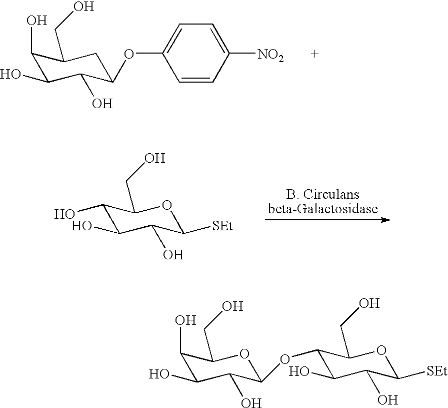

The delivery moiety of the prodrug may be selected in combination with a desired enzyme and a desired drug to deliver many appropriate drug moieties to the chosen site, such that the prodrug cannot be more readily cleaved to form the active parent drug by any other enzyme.

Preferred delivery moieties (caps) for the prodrug are selected from carbohydrate for example rhamnopyranose or variants thereof that may incorporate rhamnopyranose and a linker that will spontaneously, chemically or enzymatically fragment to release the drug, and other non-carbohydrate type caps that are degraded by the enzyme of choice.

In a further aspect of the invention there is provided a class of novel glycosylated conjugates of natural or synthetic rhamnopyranosidase enzymes, preferably glycosylated with one or more groups selected from one or a combination of the known saccharide subunits, for example the groups of mannose, galactose glucose, fucose, N-acetylglucosamine, rhamnose, saccharides and glycoconjugates thereof as hereinbefore defined as a simple monomeric glycosyl conjugate or as a complex di, poly or dendrimeric glycosyl conjugate having a suitable di, poly or dendrimeric scaffold moiety such as a di, poly or dendrivalent atom or group, and a method for the preparation thereof.

Novel enzyme conjugates include

N-WT [symbol] GalIME

Wild type naringinase modified by IME-thiogalactoside reagent

N-WT [symbol] ManIME

Wild type naringinase modified by I-thiomannoside reagent

N-WT [symbol] dendriGalIME

Wild type naringinase modified by IME-dendrithiogalactoside reagent

N-DG [symbol] ManIME

Deglycosylated naringinase, reglycosylated using IME-thiomannoside reagent

N-WT [symbol] GlyIME

Wild type naringinase modified by IME-thioglycoside reagent

N-WT [symbol] dendriGlyIME

Wild type naringinase modified by IME-dendrithioglycoside reagent

N-DG [symbol] GlyIME

Deglycosylated naringinase, reglycosylated using IME-thioglycoside reagent

N-DG [symbol] dendriGlyIME

Deglycosylated naringinase reglycosylated using IME-dendrithioglycoside reagent

The conjugates may be prepared by known chemical or biological techniques. Preferably chemical glycosylation is by reductive amination or IME-thioglycosidation, allowing the attachment of mannose, galactose and other glyco-residues as hereinbefore defined to the surface of the enzyme structure by known means. Biological techniques include enzyme synthesis, and enzyme mutagenesis, inhibiting or overexpressing an enzyme to prevent or promote a particular linkage type may be envisaged.

In a further aspect of the invention there is provided a novel method for the synthesis of glycosylated enzyme conjugates, comprising in a first stage deglycosylation of the naturally occurring (WT) or synthetic enzyme according to the scheme:
E-WT+reagent→E-DG (i)
wherein the reagent is suitably a competing enzyme, such as Endo-H and the like
followed in a second stage by protein glycosylation
E-DG+(c)I→E-DG [symbol] (c)I (ii)
with carbohydrates and linkers of formula I (Ia-Id) as hereinbefore defined using known techniques as hereinbefore defined.

Preferably, because it is thought that naringinase is already glycosylated, the method comprises a process of deglycosylation and reglycosylation, with the aim of allowing greater selectivity of enzyme delivery to sites of interest in the body, for example producing deglycosylated naringinase reglycosylated with linker I such as IME dendrithiogalactoside.

Specifically by removal of any competing glycosyl units which may have the undesirable effect of directing the enzyme to a distinct lectin specific to the competing unit, or by removal of existing similar glycosyl units in order to control the ratio of glycosyl units for controlled binding affinity, or by removal of more sterically hindered glycosyl units to improve the access and lectin binding affinity at the desired target.

Deglycosylation is by known methods for example N-WT [symbol] N-DG (or N-DG) Wild type naringinase is obtained by deglycosylation using the enzyme Endo-H.

Glycosylation may be by any known means, such as chemical modification via the linkers Ia-Id as hereinbefore defined (reductive amination^{vii}, the IME route^{viii}, N-acetyl route, 5-nitropyridine-2-sulfenyl route^{xii}, cysteine specific methanethiosulphonate route^{xi}, phenylisothiocyanate modification of lysine^{ix}, lysine specific diethyl squarate linking^{x} and the like, as illustrated in Table X), glycopeptide assembly, biological techniques, by gene expression or enzyme inhibition, or enzyme synthesis.

Preferably glycosylation is by the IME route from cyano alkyl heteroamide III as a single stage process
Rv′N(H)vCO(CH₂)nY(CH₂)nCN+MeO—→ III
Rv′N(H)vCO(CH₂)nY(CH₂)nC(═NH)OMe+H2N-(e)→ IIIa
Rv′N(H)vCO(CH₂)nY(CH₂)nC(═NM)N-(e) IIIb
wherein Y is S; and R is a hydrocarbyl group containing heteroatoms selected from a carbohydrate having an amine group, and optionally a linker as hereinbefore defined for Ia or Ia′; for example using a modified precursor of formula Ia′ according to Scheme Ia′ as hereinbefore defined.

In the case that the enzyme is not suited for any of the above techniques it may be modified by known means to introduce the appropriate initiating group, for example a modified naringinase enzyme may comprise synthetically introduced cysteine residues, which will allow the use of methane thiosulfonate reagents for glycosylation.

There is provided a class of deglycosylated enzymes of Formula IV
E-DG IV
obtained as hereinbefore defined, wherein E is preferably a non-mammalian enzyme with no mammalian equivalent selected from the rhamnopyranosidases, beta-lactamases, penicillin V amidases; or is an enzyme with mammalian equivalent selected from beta-glucuronidases; or is a mammalian enzyme selected from alpha-galactosidases and alkaline phosphatases.

There is also provided a class of novel lectin-directed glycosyl IME precursors of Formula III or IIIa as hereinbefore defined, preferably glycodendrimers wherein v′ is 2 and v″ is 1 or 2 whereby and v is 1 or 0, for conjugation with the enzyme and a novel synthesis thereof.

The kit and method may be used for delivery of any desired known or novel prodrugs for example to inhibit an enzyme's activity, preventing the production of unwanted metabolites at a particular site; to influence, ie increase or decrease, receptor response to the presence of hormones and neurotransmitters or to control the transport ability of a membrane—i.e. the range of compounds that can be transported across a particular membrane.

Prodrugs may be for either structurally specific or non-specific drug types, preferably specific whereby charge distribution and drug shape are complementary to generally three dimensional receptors in the body, of specific shapes and charge distributions, and will be readily accepted by the receptor, and so mimic the natural receptor/molecule interaction. EC₅₀ values signify the concentration required to evoke a 50% response relative to the maximum potential effect.

The drug may be selected for targeting to any desired organ and tissue types for example the liver, extrahepatic tissue or organs, the lungs, the gastrointestines or skin, where the drug is known to be metabolised.

Known prodrugs which may be delivered by the system of the invention include rhamnosides of any amine or alcohol or thiol where modification of this group reduces effects e.g., N-linked rhamnoside of methotrexate as an example of an anticancer compound, rhamnoside of vancomycin as an example of a proantibiotic, rhamnoside of doxorubicin, which has a free amino group suitable for coupling to para hydroxyphenoxy acetic acid, which is hydrolysed by a glycopenicillin V amidase conjugate, Verbascoside which is a phenylethyl glycoside with antiviral, antibacterial and antitumour properties, also an inhibitor of aldose reductase and protein kinase C, commercially available by a twenty step synthesis
Representation 2: Verbascoside 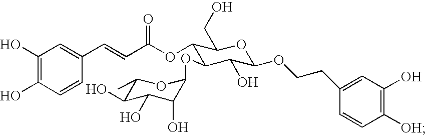
and phenolic glycoside^{xvii} 1-(α-L-rhamnosyl(1-6)-β-D-glucopyranosyloxy)-3,4,5-trimethoxybenzene (Representation 3) obtained from methanolic bark extract of Ravensura anisata Danguy (Lauraceae) from Madagascar which may be synthesised according to the invention by a simple enzyme synthesis using limited protection/deprotection and an α-rhamnopyranosidase.
Representation 3: Rhamnose-Containing Phenolic Glycoside 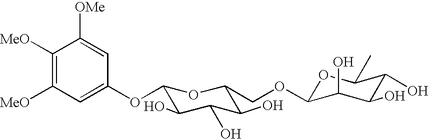
and 1-O-galloyl-α-L-rhamnose (Representation 4), a natural antibiotic isolated from the leaves of Acer rubrum L., an eastern Canadian red maple.^{xviii}
Representation 4

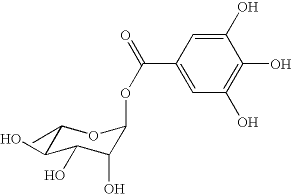

Drugs may include but not be limited to compounds acting to treat the following:

Infections such as antiviral drugs, antibacterial drugs, antifungal drugs, antiprotozal drugs, anthelmintics;

Cardiovascular system such as positive inotropic drugs, diuretics, anti-arrhythmic drugs, beta-adrenoceptor blocking drugs, calcium channel blockers, sympathomimetics, anticoagulants, antiplatelet drugs, fibrinolytic drugs, lipid-lowering drugs;

Gastro-intestinal system agents such as antacids, antispasmodics, ulcer-healing, drugs, anti-diarrhoeal drugs, laxatives, central nervous system, hypnotics and anxiolytics, antipsychotics, antidepressants, central nervous system stimulants, appetite suppressants, drugs used to treat nausea and vomiting, analgesics, antiepileptics, drugs used in parkinsonism, drugs used in substance dependence;

Malignant disease and immunosuppression agents such as cytotoxic drugs, immune response modulators, sex hormones and antagonists of malignant diseases;

Respiratory system agents such as bronchodilators, corticosteroids, cromoglycate and related therapy, antihistamines, respiratory stimulants, pulmonary surfactants, systemic nasal decongestants;

Musculoskeletal and joint diseases agents such as drugs used in rheumatic diseases, drugs used in neuromuscular disorders; and

Immunological Products and Vaccines

Prodrugs which may be delivered according to the invention include conjugates of a desired drug and a natural or synthetic carbohydrate or other activity suppressing cap as hereinbefore defined, specifically to a class of prodrug conjugates incorporating a natural or synthetic glycosyl group selected from an α-rhamnopyranosidic group, galactoside group, peptide group, glycoside group and other carbohydrate residues as hereinbefore defined and the like; or a natural or synthetic non glycosyl group selected from ester groups and the like; and other non-natural caps which may be processed by a glycoconjugate of the invention in the same way to release drug.

The prodrug is commercially available or is suitably prepared by any known means or in novel manner by enzyme synthesis preferably using the enzyme corresponding to the glycoconjugate. The use of enzymes to create the cap-to-drug linkage indicates by the principle of microreversibility that the same enzyme will also catalyse its cleavage under appropriate conditions.

It is an advantage that rhamnose type “caps” have been found to be stable to protease activity, by incubation in bacteria after which they have been found to be intact and the activation mechanism to release drug to be intact. In the case that the prodrug is capable of being hydrolysed by a native enzyme, it may be possible to coadminister with a suitable inhibitor, specific to that enzyme, to ensure prodrug delivery is targeted.

In a further aspect, the invention provides a prodrug of a class comprising prodrugs having carbohydrate cap as hereinbefore defined selected from prodrug conjugates incorporating a natural or synthetic glycosyl group selected from an α-rhamnopyranosidic group, galactoside group, peptide group, glycoside group and other carbohydrate residues as hereinbefore defined and the like, wherein the prodrugs have been obtained by enzyme synthesis with use of the corresponding non-mammalian occurring natural or synthetic enzyme selected from a rhamnopyranosidase enzyme such as naringinase, galactosidase, peptidase, glycosidase and the like or other enzyme as hereinbefore defined; or incorporating a natural or synthetic non glycosyl group selected from ester groups and the like wherein the prodrugs have been obtained by enzyme synthesis with use of a non-mammalian occurring natural or synthetic enzyme such as a peptidase.

Enzyme synthesis of prodrug is suitably by known means by contacting the selected drug in reactive form, ie as an acceptor of the selected cap, the selected cap donor and any other reagents in the presence of a suitable enzyme.

Preferably enzyme synthesis takes place in organic cosolvent to reduce competition between the glycosyl acceptor (drug) and water as nucleophile, by reducing the number of available water molecules.

Enzyme kinetic studies illustrate the effectiveness of an enzyme to carry out a particular transformation. Preferably the prodrug comprises a tracer group such as a chromophoric group, a radioactive moiety or the like whereby uptake of glycosyl groups in the drug and delivery of the drug in vivo may be monitored by depletion or production of that group. Tracer group are selected from those which will spontaneously fragment eg carbonates, carbamates, para-alkoxybenzyl under suitable conditions of pH, catalysis or that afford some form of response to chemical or biological stimulus that can be monitored or exploited in an environment specific release. A tracer group may be traceable in its intact or fragmented form. A chromophoric group or fragment may be traced by UV/visible spectrophotometry or a radioactive group such as I¹¹³⁵ by scintillation counting as known in the art and the like.

There is further provided the novel enzyme synthesis of alpha-rhamnose prodrugs as hereinbefore defined.

In enzyme synthesised glycosylation the peptide sequence affects glycosylation by making potential glycosyl acceptors unavailable, by folding into a pocket or side-chain hindrance. The type and amount of enzyme also affects glycosidation patterns. Accordingly there is an advantage to employing the corresponding technique both to make and to break the prodrug bond.

Although α-L-rhamnosidase enzymes are known from a number of industrial and research applications to hydrolyse terminal non-reducing α-L-rhamnose residues in α-L-rhamnosides.^{xix} (It should be noted that α-L-rhamnopyranosidases (E.C. number 3.2.1.40) are found in a number of different organisms, sometimes with slightly different activities), they have not previously been used to glycosylate prodrugs.

There is further provided a method of treatment comprising administering a lectin-directed glycoconjugate and a corresponding prodrug as hereinbefore defined; the method allows reduced side effects and improved specificity allowing reduced dosage quantity.

Suitably the conjugate and prodrug are adapted for administration by any appropriate means such as oral, parenteral, inhalation, topical, intravenous, rectal etc. The conjugate is adapted for administration and predetermined circulation period, for example of the order of up to 30 minutes such as 10 minutes, allows it to circulate and target the desired cells, by lectin recognition, and to clear the system in all other locations. The prodrug is adapted to be administered separately and circulates until it locates the enzyme. At this point prodrug cleavage occurs with release of drug. Preferably the enzyme conjugate remains in place for a plurality of prodrug doses.

There is further provided a dosing regime comprising a period for glycoconjugate dosing and one or more sub periods for prodrug dosing. The dosing regime may be reliant on high affinity of conjugate for lectin, and an abundance of prodrug cleavage sites, in the case that the drug is released by association of the “cap” with the enzyme, or alternatively the “cap” is released as a free entity and is cleared from the system independently of the enzyme.

There is also provided a novel composition comprising a glycosylated rhamnopyranosidase together with pharmaceutically acceptable excipients, carriers and the like suitable for mammalian, preferably human administration.

There is also provided a novel composition comprising a prodrug obtained by enzyme synthesis to introduce a cap as hereinbefore defined together with pharmaceutically acceptable excipients, carriers and the like suitable for mammalian, preferably human administration.

There is also provided the novel medical use of a glycosylated rhamnopyranosidase in the treatment of a mammal, preferably in the treatment of a human.

The inventor has devised a novel method for obtaining pure rhammopyranosidase enzyme, in particular pure naringinase enzyme. The purification method comprises dialysing a crude preparation of enzyme, subjecting the product of the dialysis to size exclusion chromatography and subjecting the product of the size exclusion chromatography to ion-exchange chromatography. This method allows 500 mg crude naringinase to yield 30 mg pure rhamnosidase activity.

The pure rhamnopyranosidase enzyme has rhamnosidase activity and is not generally contaminated by detectable glucosidase activity. In a composition containing pure rhamnopyranosidase enzyme, the enzyme may constitute e.g. more than 80%, more than 90%, more than 95%, more than 98% or more than 99% of the protein in the composition. A composition containing the enzyme may give a single band in 10% SDS-PAGE. The pure enzyme may be wild-type, deglycosylated or modified with a carbohydrate so as to produce a lectin-directed glycoconjugate of the invention.

### EXAMPLES

The invention is now illustrated in non-limiting manner with reference to the following examples.

Melting points were recorded on Gallenkamp melting point apparatus. Proton nuclear magnetic resonance (NMR) spectra were recorded, unless otherwise stated, at 200 MHz on a Varian Unity 200 spectrometer. All chemical shifts are quoted on the δ scale using residual solvent as standard. Mass spectra were recorded by the Durham University mass spectrometry using electrospray, unless otherwise stated. Microanalysis was performed by the microanalysis service at Durham University. Thin layer chromatography was carried out on aluminum sheets coated with silica gel 60 F₂₅₄. Plates were developed using an ammonium molybdate dip, or a methanol/sulfuric acid dip.

### BRIEF DESCRIPTION OF THE DRAWINGS

FIG. 1: 10% SDS-PAGE of wild-type naringinase (N-WT) subjected to freezing (lane 2) or freeze-drying (lane 3). The left-hand lane contains molecular weight markers and lane 1 contains a sample of N-WT that was not subjected to any treatment (i.e. not subjected to freezing or freeze-drying).

FIG. 2: comparison of absorbance at 280 nm with rhamnosidase activity and glucosidase activity in fractions of N-WT from a BioGel™ P-2 purification column.

FIG. 3: comparison of absorbance at 280 nm with rhamnosidase activity and glucosidase activity in fractions of N-WT from a BioGel™ P-100 purification column.

FIG. 4: 10% SDS-PAGE of fractions of N-WT from a BioGel™ P-100 purification column. The left lane contains molecular weight markers.

FIG. 5: comparison of absorbance at 280 nm with rhamnosidase activity and glucosidase activity in fractions of N-WT from a BioGel™ P-100 purification column.

FIG. 6: comparison of absorbance at 280 nm with rhamnosidase activity and glucosidase activity in fractions of N-WT from a Millipore Vantage-L™ purification column.

FIG. 7: comparison of absorbance at 280 nm with rhamnosidase activity and glucosidase activity in fractions of N-WT from a DEAE Sepharose™ purification column.

FIG. 8: comparison of absorbance at 280 nm with rhamnosidase activity and glucosidase activity in fractions of N-WT from a Sephadex G25™ purification column.

FIG. 9: 10% SDS-PAGE of various forms of N-WT.
Lane 1-molecular weight markers
Lane 2-wild-type naringinase (N-WT)
Lane 3-N-WT-GalIME
Lane 4-N-WT-ManIME
Lane 5-deglycosylated naringinase (N-DG)
Lane 6-N-DG-GalIME
Lane 7-empty
Lane 8-N-WT
Lane 9-N-WT-dGalIME

FIG. 10: enzyme activity of N-WT at varying pH.

FIG. 11: enzyme activity of N-DG at varying pH.

FIG. 12: activities of wild-type and variously modified forms of naringinase.

FIG. 13: radio-imaging of rabbits treated with radio-labeled wild-type naringinase modified with GalIME (N-WT□GalIME), either alone (top panels) or with the blocker asialofetuin (AF) (bottom panels).

FIG. 14: liver retention of N-WT□Gal over 120 min, either alone or with the blocker asialofetuin (AF).

FIG. 15: liver retention of N-WT□dGalIME over 120 min, either alone or with the blocker asialofetuin (AF).

FIG. 16: extinction coefficient of p-nitrophenol (NP) in tritosome assay conditions (blanked to zero).

FIG. 17: tritosomal stability of N-WT.

FIG. 18: panel (a) shows a section from liver of N-DG□dGalIME dosed animal under phase imaging conditions and panel (b) shows the same section in the same orientation under fluorescence imaging conditions with a substrate that generates a fluorescent product.

### A Preparation of Glycosylation Precursors

### Example A1

### Novel Synthesis of Cyano Precursor to Galactose Dendrimer IME

The synthesis was first conducted to prepare N-Benzyl-4-cyanomethylsulfanyl-butyramide

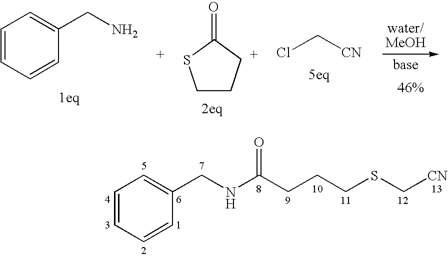

Benzylamine (0.55 ml, 5.0 mmol), γ-thiobutyrolactone (0.87 ml, 10.0 mmol) and chloroacetonitrile (1.58 ml, 25 mmol) were added to an aqueous solution of sodium hydrogen carbonate (30 ml, 0.5M) and methanol (25 ml) in a round bottom flask fitted with reflux condenser, magnetic stirrer bar and inert atmosphere. The mixture was heated overnight at 50° C.

The methanol was removed in vacuo, and the resultant aqueous layer extracted with chloroform (3×60 ml). The organic layers were combined and washed with hydrochloric acid (3×60 ml, 1M) then dried over magnesium sulfate. This solution was filtered, and all solution removed in vacuo.

The product oil was purified by flash chromatography (3:1 EtOAc:Hexane+1% Et₃N).

Yield=0.57 g, 46%. ¹H-NMR (250 MHz CDCl₃) δ 2.04 (quin, 2H, H¹⁰), 2.36, 2.80 (2×t, 2×2H, H⁹ & H¹⁰), 3.28 (s, 2H, H¹²), 4.43 (d, 2H, H⁷), 5.85 (s, 1H, NH), 7.26-7.31 (5×s, 5H, Ph)

### Synthesis of Cyano Precursor Glycodendrimer-IME Reagent

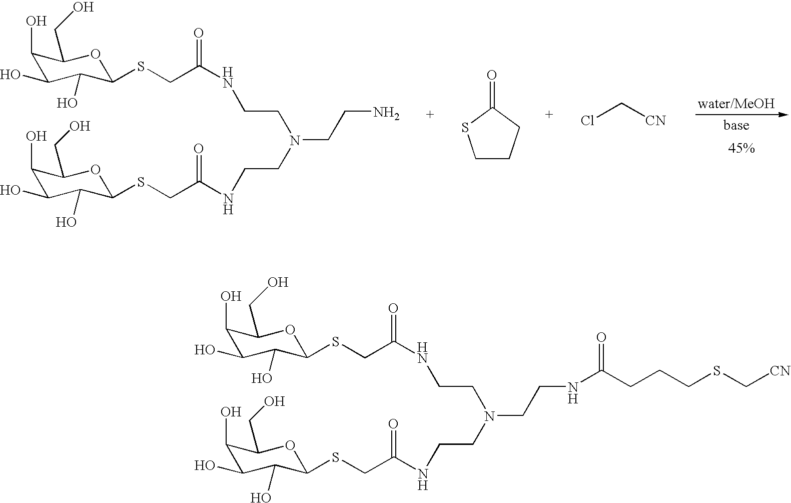

Bis {N-[2-(1-thio-β-D-galactopyranosyl)ethanoyl]aminoethyl}amine (98.9 mg, 0.16 mmol), γ-thiobutyrolactone (0.14 ml, 1.6 mmol) and chloroacetonitrile (0.20 ml, 3.2 mmol) were added to an aqueous solution of sodium hydrogen carbonate (1 ml, 0.5M) and methanol (1 ml) in a round bottom flask fitted with reflux condenser, magnetic stirrer bar and inert atmosphere. The mixture was heated for 24 hours at 50° C.

The mixture was neutralized with 2M HCl, concentrated in vacuo and purified by flash chromatography (CHCl₃:MeOH:H₂O:Et₃N 60:35:7:1) to yield a product, which was taken to dryness in vacuo. The product was then loaded onto Dowex 50W2-200 (H⁺) in water:methanol 1:1 and eluted with 10% NH₃ solution. The product dried by freeze-drying.

Yield=54.7 mg, 45%. ¹H NMR (300 MHz, CD₃OD) ˜1.25 (t, 4H, CH₂CN), 2.18 (q, 2H), 2.5 (t, 2H), 2.7 (t, 4H), 2.9 (dd, 2H), 3.35 (m, 24H), 4.47 (d, 2H, anomeric H). Mass confirmed by ES-MS.

### Ex A 2 Preparation of 1,2,3,4,6-Penta-O-acetyl β-D-mannopyranoside

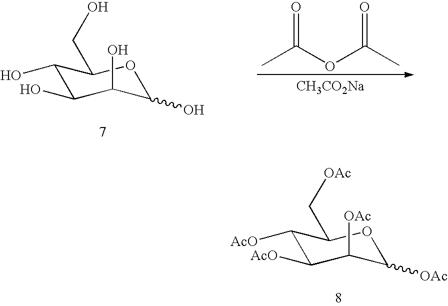

Sodium acetate (10 g, 121.9 mmol) was suspended in acetic anhydride (70 ml), heated until gently refluxing and then removed from the heat. Small portions of D-mannose (total 10 g, 55.49 mmol) were added at a rate that maintained reaction. A heat gun was used to maintain reaction temperature where necessary. After the reaction had subsided, as detected by a lack of bubbling, the orange coloured mixture was returned to the heat and refluxed for 1 hour. The mixture was allowed to cool and was then poured into ice water (200 ml) and left to stand, with occasional stirring, for 3 hours. This yielded a dark brown mixture with little separation between organic and aqueous layers. Extraction by ethyl acetate (5×150 ml) followed by evaporation in vacuo yielded a dark brown syrup. Purification by flash column chromatography (3:1, ethyl acetate:hexane) yielded an orange oil, (8).

Yield: 19.83 g, 91.6%. Elemental analysis: Theoretical: C 49.2%; H 5.68%; N 0.0%; Actual: C 49.49%; H 6.09%; N 0.00%. ¹H NMR (200 MHz, CDCl₃, α:β 10:1) δ 1.72, 1.75, 1.78, 1.79, 1.80, 1.81, 1.90, 1.92 (s×8, 3H×10, Ac×10, 2 anomers), 3.82 (dd, 1H, J=5, J=12), 4.00 (dd, 1H, J=5, J=12) 5.0 (m, 2H), 5.79 (d, 1H, J=2, H−1, α), 5.92 (d, 1H, H−1, β). [α]_{D}^{23.3}=+37.895 (CHCl₃, c=2.66). Mpt°=93.2-95.2° C.

### Ex A3 Preparation of 1,2,3,4,6-Penta-O-acetyl β-D-galactopyranoside

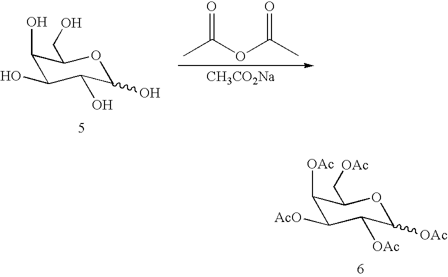

Sodium acetate (10 g, 121.9 mmol) was suspended in acetic anhydride (70 ml), heated until gently refluxing and then removed from the heat. Small portions of D-galactose (total 10 g, 55.49 mmol) were added at a rate that maintained reaction. A heat gun was used to maintain reaction temperature where necessary. After the reaction had subsided, as detected by a lack of bubbling, the orange coloured mixture was returned to the heat and refluxed for 1 hour. The mixture was allowed to cool and was then poured into ice water (200 ml) and left to stand, with occasional stirring, for 3 hours. This yielded a dark brown mixture with little separation between organic and aqueous layers. Extraction by ethyl acetate (5×150 ml) followed by evaporation in vacuo yielded a dark brown syrup. Purification by flash column chromatography (3:1 ethyl acetate:hexane) yielded an orange oil, (6).

Yield 19.27 g, 89.0%. Elemental analysis: Theoretical: C 49.2%; H 5.68%; N 0.0%; Actual: C 49.18%; H 5.71%; N 0.0%. ¹H NMR (200 MHz, CDCl₃) δ 2.00 2.03 2.05 2.13 2.17 (s×5, 3H×5, Ac×5), 4.10 (m, 3H, H−5, H−6, H−6′) 5.08 (dd, J 3 Hz J 10 Hz, 1H, H-2), 5.36 (m, 2H, H-3, H−4), 5.70 (d, J=8, 1H, H−1).

### Ex A4 Preparation of 2,3,4,6-Tetra-O-acetyl-α-D-mannopyranosyl bromide

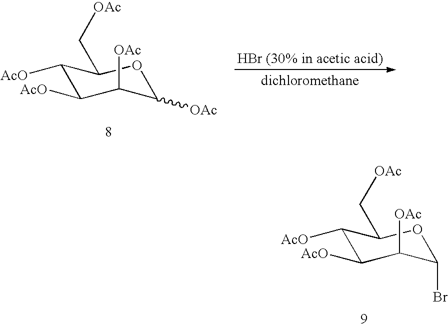

1,2,3,4,6-O-pentaacetyl D-mannopyranose, (3.69 g, 9.45 mmol) was dissolved in dry dichloromethane (130 ml) and stirred in a 250 ml round bottom flask at 0° C. under a nitrogen atmosphere. Hydrogen bromide (20 ml, 30% in acetic acid) was added slowly, and the mixture stirred at 0° C. for 15 minutes before warming to room temperature. The reaction was followed by thin layer chromatography, using 1:1 ethyl acetate:hexane (R_{F} starting material=0.3, product=0.5).

After 4 hours the green/brown mixture was poured over ice and a colour change to white was observed. This solution washed with a saturated solution of sodium hydrogen carbonate (3×120 ml) and then with water (2×120 ml). The organic layer was dried over magnesium sulfate, filtered, and taken to dryness in vacuo, yielding a clear, viscous oil (9). P Yield 2.38 g, 61.3%. Elemental analysis: Theoretical: C 40.90%; H 4.66%; N 0.0%; Actual: C 40.31%; H 4.59%; N 0.00%. ¹H NMR (250 MHz, CDCl₃) δ 1.95 2.01 2.04 2.11 (s×4, 3H×4, Ac×4), 4.07 (dt, H×1, H−5), 4.15 (m, H×1, H−4), 4.25 (dd, H×1, H−3), 5.31 (m, H×2, H−6H′−6), 5.65 (dd, H×1, H−2), 6.23 (d, H×1, H−1). ESMS Required peak 433/435, observed peak 433/435. [α]_{D}^{23.3}=+123.757 (CHCl₃, c=1.81).

### Synthesis of 2-Imino-2-methoxyethyl 1-Thioglycosides

### Ex A5 Synthesis of 2-S-(2,3,4,6,-Tetra-O-acetyl-β-D-galactopyranosyl)-2-thiopseudourea hydrobromide (TPU Derivative of Galactose)

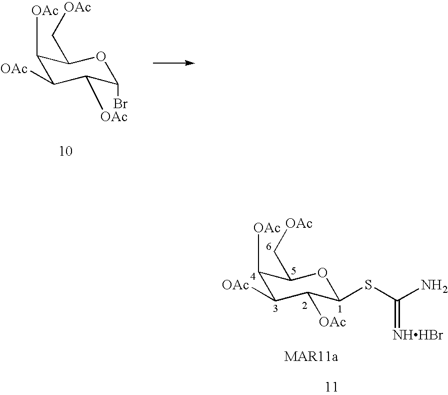

Acetobromo-α-D-galactose (16.448 g, 40 mmol) and thiourea (3.958 g, 52 mmol) were suspended in dry acetone (15 ml) in a 250 ml round bottom flask fitted with a magnetic stirrer and reflux condenser, under a nitrogen atmosphere. The mixture was refluxed for 2 hours and the reaction followed by tlc, reduced in volume (30 ml) in vacuo and chilled for 3 hours. The resulting white crystals (11) were filtered, washed with cold acetone and dried.

Yield: 14.24 g, 73.1%. Elemental Analysis: Theoretical: C 36.97%; H 4.76%; N 5.75% Actual: C 38.79%; H 5.23%; N 5.28%. ¹H NMR (CDCl₃, 199.99 Hz): δ =1.982-2.186 (s, 4 OAc), 4.147-4.179 (s, 2H⁶), 4.46 (d, H²), 5.2 (dd, H²), 5.379 (td, H⁵), 5.486 (dd, H³), 5.529 (dd, H⁴), 8.294 (s, NH₂), 9.445 (s, NH₂). ¹³C NMR (CDCl₃, 62.90 MHz): δ=20.4-20.8 (4× Acetyl-Me), 30.9 (imidate carbon), 61.2-82.0 (6× ring carbons), 169.0-170.8 (4× Acetyl carbonyl). [α]_{D}^{21.6}=+0.6 (CHCl₃, c=1.0). Mpt°=174.7-176.5.

### Ex A6 Synthesis of Cyanomethyl Per-O-acetyl-1-thiogalactopyranoside

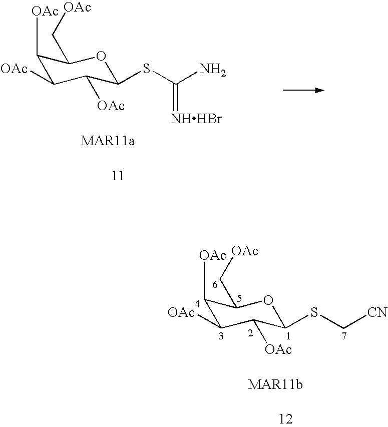

11 (3.7 g, 7.6 mmol) was stirred with dry acetone (20 ml) and water (20 ml) was added. Sodium bisulfite (2.0 g, 38 mmol), potassium carbonate (1.6 g, 11.5 mmol) and chloroacetonitrile (2.5 ml, 40 mmol) were then added in order, and the mixture stirred at room temperature for 3 hours. This mixture was then added to ice water (40 ml) and stirred for 2.5 hours at 4° C. Some acetone (20 ml) was removed in vacuo and the resulting mixture chilled overnight in a refrigerator. The red gum product was extracted with chloroform (2×40 ml) treated with decolourising charcoal, and filtered through a Celite column and the extract washed with sodium chloride solution (3×40 ml, 1M), and the resulting organic layer was dried over anhydrous sodium sulfate. The chloroform solution was then evaporated to dryness in vacuo and the product gum recrystallized from hot ethanol, yielding a white crystalline solid.

Yield: 1:545 g, 50.4%. Elemental Analysis: Theoretical: C 47.65%; H 5.25%; N 3.47% Actual: C 47.61%; H 5.27%; N 3.46%. ¹H NMR (CDCl₃, 199.99 Hz): δ=1.993-2.169 (s, 4 OAc), 3.492 (s, 2H⁷), 4.0 (dd, H²), 4.135-4.162 (dd, 2H⁶), 4.6985 (d, H¹), 5.1 (dd, H³), 5.252 (td, H⁵), 5.466 (dd, H⁴). ¹³C NMR (CDCl₃, 62.90 MHz): δ=14.4 (C-7), 20.5-20.7 (4× Acetyl-Me), 61.3-82.2 (6× ring carbons), 115.7 (cyano-carbon), 169.8-170.3 (4× Acetyl carbonyl). [α]_{D}^{21.7}−56.96 (CHCl₃, c=0.95). Mpt°=93.2-95.2° C.

### Ex A7 Synthesis of 2-Imino-2-methoxyethyl 1-thiogalactoside

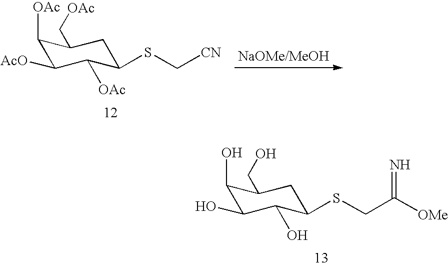

A solution of cyanomethyl per-O-acetyl-1-thiogalactopyranoside (0.4805 g, 1.19 mmol) in dry methanol (20 ml) was added to a methanolic solution of sodium methoxide (0.01M, 30 ml) and stirred at room temperature under a nitrogen atmosphere for 48 hours. The methanol was removed in vacuo, and protein conjugation attempted immediately.

### Ex A8 Preparation of 2-S-(2,3,4,6-tetra-O-acetyl α-D-mannopyranosyl)-2-thiopseudourea hydrobromide

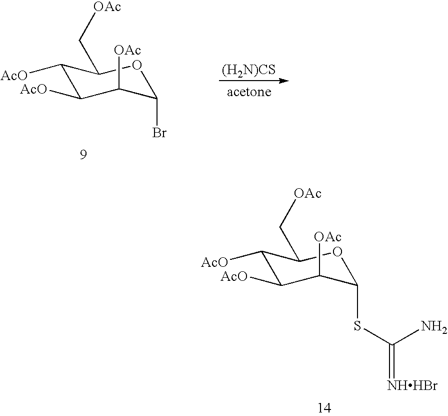

Acetobromo-α-D-mannopyranoside (2.36 g, 5.7 mmol) and thiourea (0.574 g, 7.54 mmol) were suspended in dry acetone (25 ml) in a 50 ml round bottom flask fitted with a reflux condenser and magnetic stirrer. The mixture was refluxed for 3 hours, 20% of the solvent was removed in vacuo, and then chilled in an ice bath for 2 hours. As no precipitation occurred, the solution was chilled in a freezer overnight. Still no precipitation was observed, so all solvent was removed in vacuo yielding a pale yellow semi-crystalline gum. Recrystallisation from hot ethyl acetate resulted in precipitation of product. After filtration and drying in vacuo, the reaction yielded a white powder (14).

Yield: 0.393 g, 14.16%. Elemental Analysis: Theoretical: C 36.97%; H 4.76%; N 5.75% Actual: C 36.85%; H 4.72%; N 5.48%. ¹H NMR (CDCl₃, 499.78 Hz): δ 2.013, 2.100 2.131 2.195 (s×4, 3H×4, Ac×4), 4.20 (dd, H×1, H−3), 4.310 (dd, H×1, H−4), 4.385 (td, H×1, H−5), 5.09 (dd, H×1, H−2), 5.313 (t, H×1, H−6), 5.430 (d, H×1, H−1), 8.231 (s, H×2, —NH₂), 9.725 (s, H×2, —NH.HBr). ¹³C NMR (CDCl₃, 62.90 MHz): δ=20.5-20.8 (4× acetyl methyl), 61.8-82.4 (6× ring carbons), 169.2-170.4 (4× acetyl carbonyl).

### Ex A9 Preparation of Cyanomethyl per-O-acetyl-1-thiomannopyranoside

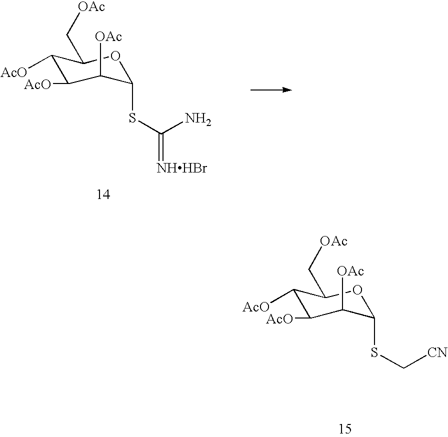

2-S-(2,3,4,6-tetra-O-acetyl α-D-mannopyranosyl)-2-thiopseudourea hydrobromide (0.37 g, 0.76 mmol) was suspended in acetone (2 ml), to which water (2 ml), sodium bisulfite (0.2 g, 3.8 mmol), potassium carbonate (0.16 g, 1.15 mmol) and chloroacetonitrile (0.25 ml, 4 mmol) were then added, in order. This mixture was stirred at room temperature for 3 hours, then poured over ice water (15 ml) and stirred at 4° C. for 2 hours. A white precipitate formed, and was filtered, washed with iced water and then dried in vacuo. Recrystallisation from hot methanol yielded a white solid (15).

Yield: 84.4 mg, 27.5%. Elemental Analysis: Theoretical: C 47.65%; H 5.25%; N 3.47% Actual: C 47.39%; H 5.22%; N 3.36%. ¹H NMR (CDCl₃, 499.78 Hz): δ 2.003, 2.065, 2.112, 2.189 (s×4, H×12, Ac×4), 3.342 (dd, H×2, —CH₂—), 4.18 (dd, H×1, H−2), 4.343 (m, H×3), 5.20 (dd, H×1, H−3), 5.34 (dd, H×1, H−4), 5.376 (t, H×1), 5.462 (d, H×1, H−1). ¹³C NMR (CDCl₃, 62.90 MHz): δ=15.8 (—S—CH₂—CN), 20.2-21.1 (4× Acetyl-Me), 62.1-82.4 (6× ring carbons), 115.9 (cyano-carbon), 169.8-171.0 (4× Acetyl carbonyl). ESMS Required peak 426, observed peak 426. [α]_{D}^{21.7}=+111.7 (CHCl₃, c=0.52). Mpt°=111.2-113.4° C.

### B Glycosylation Reactions: Preparation of Glycoconjugate

### Ex B1 Glycosylation of Naringinase Using IME-Thiogalactoside

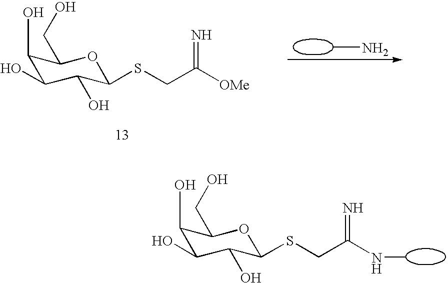

13 (1.19 mmol) was kept in its round bottom flask, a solution of naringinase (0.1070 g in 20 ml 0.25M sodium borate—pH8.5) was added. And the mixture was stirred under a nitrogen atmosphere for 48 hours. After this time, purification by Biogel P-2 was carried out (2×60 cm, 0.1M NaCl, pH4.8) 0.1M sodium chloride solution, pH 4.8, as eluant. Presence of protein was detected by spectroscopy (absorbance at 280 nm) and the relevant fractions were combined and freeze dried.

### Ex B2 IME-Thiomannosidation of WT Naringinase

1.1

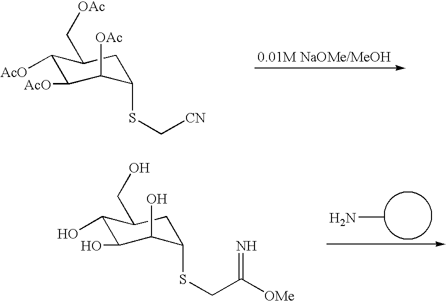

One major aspect of this work to introduce specific carbohydrate moieties to the surface of naringinase, the α-rhamnopyranosidase, with the aim of being able to specifically deliver naringinase, to particular cell types using lectin-carbohydrate interactions. This requires the investigation of a number of glycosylation techniques, namely reductive amination and the use of IME-thioglycosides. Both techniques rely on the presence of free amino groups within the protein structure—and that these amino groups are not hindered by the presence of large sterically prohibitive groups within the protein structure.

Naringinase was treated with IME-thiomannoside, in an attempt to modify any accessible amino groups on the surface of the protein. The cyanomethyl per-O-acetyl-1-thiomannoside (12) was treated with a methanolic solution of sodium methoxide, yielding the IME-thiomannoside (13). Once all solvent had been removed from the flask in which the IME-thiomannoside had been prepared, a solution of naringinase with sodium tetraborate buffer (pH 8.5) was added, and stirred under an inert atmosphere for two days.

BioGel P2 size exclusion chromatography (2×60 cm, 0.1M NaCl, pH 4.8) was used to purify the naringinase, with aliquots tested by absorption at 280 nm to detect presence of protein. Relevant fractions were combined and freeze-dried before being tested for enzyme activity. 12.5% SDS PAGE was used to determine any change in mass brought about by this method.

### Ex B3 Deglycosylation of Enzyme

Purification of Endoglycosidase H

A stock solution of Endoglycosidase H (EndoH) (1 ml, 10 mg/ml) was diluted to 20 ml with deionised water and then dialysed using Visking tubing (12-14 kDa MWCO) against deionised water for 24 hours, with water changes at 1, 6 and 18 hours. The resultant solution was freeze-dried yielding a white powder. Purity was assessed by gel electrophoresis (SDS PAGE), in the gel a reduction in molecular weight of around 25-30 kDa is clearly observed (the difference between bands 2 and 5).

Deglycosylation of N-WT (i.e. Preparation of N-DG)

Two comparable methods have been tested, in an attempt to assess the effect of scale on reaction success.

A solution of N-WT (5 ml, 10 mg/ml in 10 mM pH6.0 orthophosphate buffer) and a solution of EndoH (25 μl, 10 mg/ml solution) were combined in a sample vial fitted with a magnetic stirrer bar and heated at 37° C. for 24 hours. The solution was transferred to a Spectrum DispoDialyser® (50 kDa MWCO) and dialysed against deionised water for 24 hours, with water changes at 1, 6 and 18 hours. The resultant solution was freeze-dried yielding a white powder. Purity was assessed by gel electrophoresis (SDS PAGE), see later.

A solution of N-WT (20 ml, 10 mg/ml in 10 mM pH6.0 orthophosphate buffer) and a solution of EndoH (100 μl, 10 mg/ml solution) were combined in a round bottom flask fitted with a magnetic stirrer bar and heated at 37° C. for 24 hours. The solution was transferred to Spectrum Cellulose Ester dialysis tubing (50 kDa MWCO) and dialysed against deionised water for 24 hours, with water changes at 1, 6 and 18 hours. The resultant solution was freeze-dried yielding a white powder. Purity was assessed by gel electrophoresis (SDS PAGE), see later.

### Ex B 4 Preparation of N-DG→GalIME

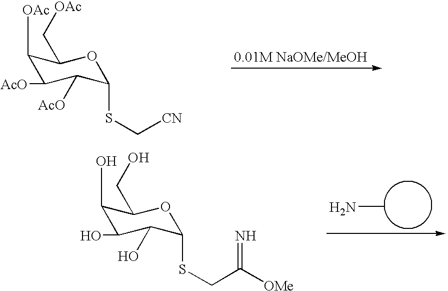

GalIME reagent (45 mg, 0.11 mmol) was dissolved in dry methanol (20 ml) in a 100 ml round bottom flask fitted with a magnetic stirrer under an inert atmosphere. A methanolic solution of sodium methoxide (30 ml, 0.01M) was added, and the mixture stirred for 36 hours at room temperature.

The contents of the flask were taken to dryness in vacuo, and a solution of N-DG (2 ml, 5 mg/ml in 0.25M sodium borate, pH8.5 corrected with cHCl) was added. The mixture was stirred under a nitrogen atmosphere for 36 hours. After this time, the solution was purified by Biogel P-2 column (2×60 cm, 0.1M NaCl, pH4.8) with presence of protein detected by spectroscopy (absorbance at 280 nm). Appropriate fractions were combined and freeze dried, resulting in a white powder. Purity was assessed by gel electrophoresis (SDS PAGE), Successful glycosylation was detected, the difference between bands 5 and 6 suggests an increase in molecular weight of around 1.5 kDa, equivalent to the addition of ten carbohydrate molecules to the protein.

Protein Purification

All naringinase samples were desalted using Sephadex PD10 desalting columns. A maximum of 25 mg protein was put on to each column, which were pre-eluted with deionised water. SDS PAGE has been employed successfully in the determination of molecular weights of all protein samples. The appropriate fractions were combined and freeze-dried.

Gel Electrophoresis

Aqueous solutions of all naringinase samples were prepared (2 mg/ml) and loaded onto 12% gels in the usual manner. The gels were run at 150V for 3 hours, removed and stained overnight, followed by destaining overnight to remove background stain and molecular weight successfully assessed for all samples, 1 BioRad SDS PAGE Protein Standard (from top: 97.4, 66, 45 (faint), 31 kDa)

2 N-WT, 3 N-WT→Gal-IME, 4 N-WT→ManIME, 5 N-WT→N-DG, 6 N-DG→GalIME.

### C Enzymatic Synthesis: Preparation of Prodrug

### Ex C1 Enzymatic Reaction Between β-Methyl Glucopyranoside and p-nitrophenyl α-L-rhamnopyranoside

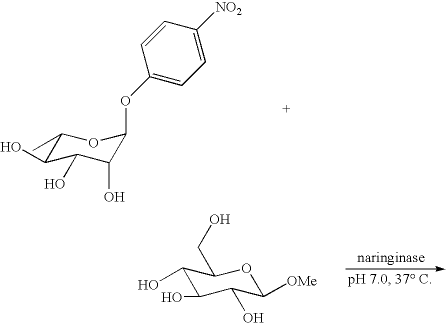

A solution of p-nitrophenyl α-L-rhamnopyranoside (3.5 mM, 0.0050 g, 1.75×10⁻⁵ mol) in orthophosphate buffer (5 ml, pH 7.0) was stirred in a 50 ml round bottom flask in a graphite bath thermostatted to 37° C. A solution of β-methyl glucopyranoside (0.0204 g, 1.05×10⁻⁴ mol) in orthophosphate buffer (3 ml, pH 7.0) was added, with naringinase (0.0147 g, 5 Units) and an inert atmosphere introduced. The reaction was followed by thin layer chromatography (1:9 methanol:ethyl acetate) and when all glycosyl donor was consumed the reaction was quenched by placing in a water bath at 100° C. for 5 minutes. All water was removed by freeze-drying.

### Ex C2 Acetylation of Enzyme Reaction

Dry pyridine (20 ml) and acetic anhydride (10 ml) were added to the crude product of MAR48, and stirred in a 50 mil round bottom flask overnight. Methanol (20 ml) was added, whilst the mixture was cooling in an ice bath, to quench the reaction, and then all solvents were removed in vacuo. The residue was dissolved in dichloromethane (15 ml) and washed with a sodium hydrogen carbonate solution (0.05M, 3×40 ml). The organic layer was dried over magnesium sulfate, filtered and all solvent removed in vacuo. This mixture was purified by flash chromatography, using a 1:1 ethyl acetate:hexane solvent system.

### Ex C3 Enzymatic Reaction Between β-Methyl Galactopyranoside and p-Nitrophenyl α-L-rhamnopyranoside

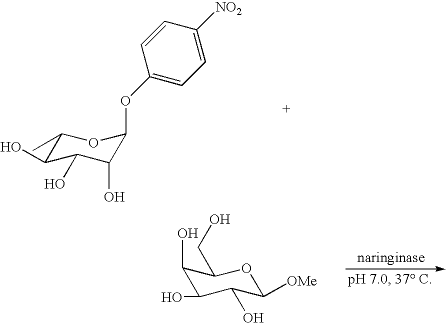

A solution of p-nitrophenyl α-L-rhamnopyranoside (3.5 mM, 0.0050 g, 1.75×10⁻⁵ mol) in orthophosphate buffer (5 ml, pH 7.0) was stirred in a 50 ml round bottom flask in a graphite bath thermostatted to 37° C. A solution of β-methyl galactopyranoside (0.0323 g, 1.663×10⁻⁴ mol) in orthophosphate buffer (3 ml, pH 7.0) was added, with naringinase (0.0147 g, 5 Units) and an inert atmosphere introduced. The reaction was followed by thin layer chromatography (1:9 methanol:ethyl acetate) and when all glycosyl donor was consumed the reaction was quenched by placing in a water bath at 100° C. for 5 minutes. All water was removed by freeze-drying.

### Ex C4 Acetylation of Enzyme Reaction

Dry pyridine (20 ml) and acetic anhydride (10 ml) were added to the crude product of MAR50, and stirred in a 50 ml round bottom flask overnight. Methanol (20 ml) was added, whilst the mixture was cooling in an ice bath, to quench the reaction, and then all solvents were removed in vacuo. The residue was dissolved in dichloromethane (15 ml) and washed with a sodium hydrogen carbonate solution (0.05M, 3×40 ml). The organic layer was dried over magnesium sulfate, filtered and all solvent removed in vacuo. This mixture was purified by flash chromatography, using a 1:1 ethyl acetate:hexane solvent system.

NMR Study:

Solutions of p-nitrophenyl α-L-rhamnopyranoside (3 mM, 200 μl) and α-methyl galactopyranoside (18 mM, 200 μl) in water were mixed in an NMR tube fitted with a capillary insert dilled with D₂O. Suppression of the water signal in the sample was achieved by pre-irradiating the sample at the appropriate water signal frequency. An aliquot of naringinase solution (20 μl, 1 mg/ml, pH 7.0 orthophosphate buffer) was added to the NMR tube and an NMR spectrum taken every two minutes for four hours.

### D Preparation of Prodrug

### Synthesis of Naringinase Substrate—p-Nitrophenyl α-L-rhamnopyranoside

The starting material p-nitrophenyl α-L-rhamnopyranoside (18) was synthesised on a gram scale employing a previously published^{xx} strategy.

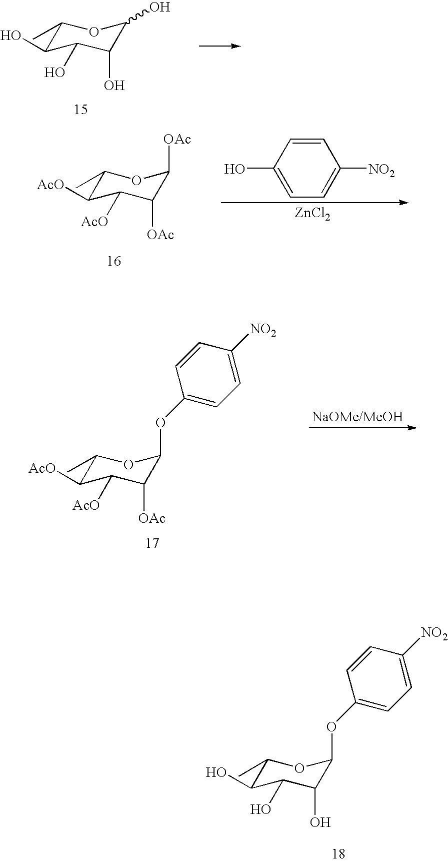

The procedure follows standard techniques of protection of L-rhamnose (15) by acetylation using acetic anhydride and a catalytic amount of sodium acetate to yield (16) in 68% yield, followed by Lewis Acid aided coupling in a “melt” reaction to form (17) in 5.54% yield. This was followed by standard methanolic deprotection to yield the desired product (18). Protection and deprotection strategies worked well, with respectable yields.

Purification of the final material by washing with water and diethyl ether allowed drying of the product on a freeze drier, which after analysis proved to be pure enough for future use. A slight yellow discolouration is due to the presence of free p-nitrophenol, which is not detected by NMR (<2%).

### Example D1 Preparation of 1,2,3,4-Tetra-O-acetyl rhamnopyranose

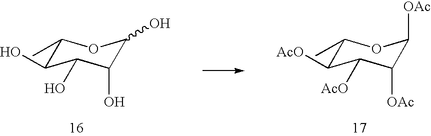

L-rhamnopyranose monohydrate (11.10 g, 60.9 mmol), sodium acetate (5.5 g, 67.0 mmol) and acetic anhydride (90 ml) were stirred in a 250 ml round bottom flask fitted with a reflux condenser, and an inert atmosphere was introduced. The mixture was heated using an oil bath with a thermostat set to 110° C. for 90 minutes. The mixture was then removed from the heat, and when cool poured over ice water (500 ml). After allowing to stand for 2 hours, this mixture was extracted with chloroform (3×100 ml) and dried overnight over calcium chloride). After filtration, all solvent was removed in vacuo yielding a pale yellow oil, and synthesis proceeded to the next stage without purification.

### Ex D2 Tri-2,3,4-O-acetyl-p-aminophenyl α-L-rhamnopyranoside

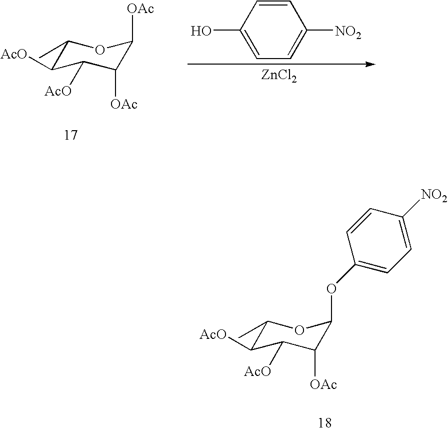

Tetra-1,2,3,4-O-acetyl-rhamnopyranose (13.76 g, 41.4 mmol, from MAR56) and p-nitrophenol (19.264 g, 138.4 mmol) were combined in 250 ml round bottom flask and heated at 120° C., under vacuum from a water pump, until all bubbling had subsided. Dry, finely powdered zinc chloride (6.88 g, 50.5 mmol) was then added to the now molten mixture, and the vacuum reapplied. The mixture turned from pale yellow to dark brown and was heated for 60 minutes at 120° C., and then cooled to 50° C. This mix was then extracted with chloroform (200 ml) and washed with a solution of sodium hydroxide (1M, 3×200 ml) and then water (5×200 ml). The organic layer was dried over magnesium sulfate and the solvent removed in vacuo.

The resultant brown gum was dissolved in refluxing methanol (120 ml), passed over prewashed activated charcoal, filtered, and left to stand overnight. As no precipitation had occurred, some solvent was removed in vacuo, and the solution placed in the freezer overnight. Pale brown crystals formed, which were recrystallised from hot methanol, and a series of crops were taken resulting in the final yield.

Yield: 0.944 g, 5.54%. ¹H NMR (CDCl₃, 199.99 Hz): δ 1.21 (d, H×1, H−1), 2.0 2.1 2.2 (s×3, H×3, Ac×3), 3.9 (dq, H×1, H−5), 5.2-5.6 (m, H×6,), 7.2 (dd, H×2, Ar—H), 8.2 (dd, H×2, Ar—H). ESMS Required peak 434, observed peak 434.

### Ex D3 Preparation of p-Nitrophenyl α-L-rhamnopyranoside

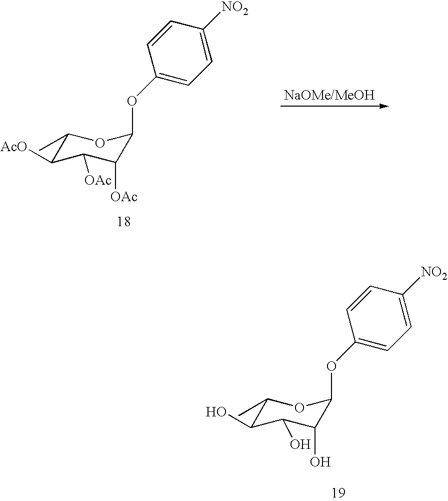

Per-O-acetyl p-nitrophenyl α-L-rhamnopyranoside (0.944 g, 2.39 mmol) was dissolved in dry methanol (25 ml) in a 100 ml round bottom flask, to which sodium methoxide (1.5 ml, 0.1M) was added. The mixture was refluxed for 15 minutes, until the reaction was complete, and then taken to dryness in vacuo. The ethanol was removed in vacuo. The resultant solid was dissolved in water, and washed with diethyl ether to remove p-nitrophenol. The water was removed by the use of a freeze dryer, yielding an off-white product.

Elemental Analysis Theoretical: C 50.53%; H 5.30%; N 4.91% Actual: C 48.92%; H 5.52%; N 5.38%. ¹H NMR (CDCl₃, 499.99 Hz): δ 1.081 (d, H×3, H-6×3), 3.386 (t, H×1,), 3.58 (dq, H×1, H−5), 3.876 (dd, H×1, H−2), 4.046 (d, H×1, H−1), 7.115 (d, H×2, Ar—H×2), 8.122 (d, H×2, Ar—H×2). [α]_{D}^{23.5}=−164.87 (MeOH, c=0.19). Mpt°=171.4-173.0° C.

### E Prodrug Release: Enzyme Kinetic Study

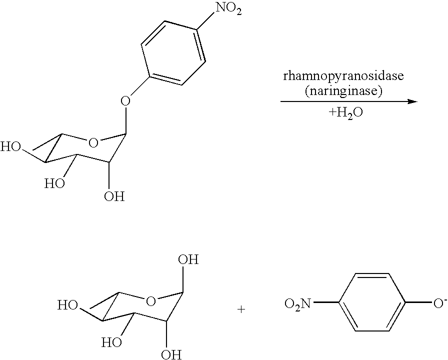

An aqueous naringinase solution (10 μl, 0.5 mgml⁻¹) was tested for catalytic activity against a range of concentrations of p-nitrophenyl α-L-rhamnopyranoside solutions (190 μl, 3.5, 3.0, 2.5, 2.0, 1.5, 1.0, 0.5, 0.25 mM in 0.2M orthophosphate buffer—pH 6.8, 7.0, 7.2).

The substrate solution was pipetted into wells on a multiwell plate, and incubated at 37° C. for 5 minutes. The naringinase solution was then added, and the absorbance read at 405 nm for 10 minutes, at 6 second intervals with shaking for 5 seconds before the start of reading, and 1 second before each individual reading.

F Enzyme Stability

Heat Stability Studies:

Prior to testing using the above procedure, the enzyme solution was incubated in a water bath at the desired temperature for the appropriate length of time.

Organic Solvent Stability Studies:

In addition to testing using the general procedure, the enzyme was introduced to the desired organic solvent (with predetermined organic/aqueous ratio) for a set period of time.

Proteolytic Stability of Naringinase

Naringinase is to be used as part of a bipartate drug mechanism and so its ability to withstand the attack of proteases is essential to the success of the system. To this end a protocol has been developed whereby a naringinase solution is treated with a 5% equivalent solution of Subtilisn Bacillus Lentis, and after various incubation periods the naringinase solution was employed in a standard kinetic assay. As deglycosylated naringinase is to be used in further reglycosylation studies, this deglycosylated naringinase was also tested in the same manner. Wild type naringinase proved to be fairly stable to protease activity, with only slight loss of activity over the 48 hour period.

Kinetic Assays

Assays showed that glycosylation does not adversely affect lectin directing activity

An aqueous naringinase solution (10 μl) was tested for catalytic activity against a range of concentrations of p-nitrophenyl α-L-rhamnopyranoside solutions (190 μl, 3.5, 3.0, 2.5, 2.0, 1.5, 1.0, 0.5, 0.25 mM in 0.2M orthophosphate buffer—pH 6.8, 7.0, 7.2).

The substrate solution was pipetted into wells on a multiwell plate, and incubated at 37° C. for 5 minutes. The naringinase solution was then added, and the absorbance read at 405 nm for 10 minutes, at 6 second intervals with shaking for 5 seconds before the start of reading, and 1 second before each individual reading.

In addition, during heat stability studies, the enzyme solution was incubated in a water bath at the desired temperature for the appropriate length of time before use.

Absorbance data was measured using a UV-vis spectrometer, converted to rate information in MS Excel and then kinetic parameters determined using GraFit 4 from Erithacus Software.

Full kinetic parameters were determined for N-WT→N-DG and N-DG→GalIME, N-WT, N-WT-GalIME and N-WT-ManIME. N-DG shows greater rate of reaction than N-WT and its ability to bind to substrates is similar resulting in a more active enzyme, attributed to removing obstructing glycosyl groups, with no change in stability or conformation.

N-WT, N-WT-GalIME and N-WT-ManIME were found to be stable at 60° C. (greater than physiological temperature) and at physiological pH

### G In Vivo Biodistribution Assessment

### Preparation of Radiolabelled Enzyme Samples

The enzyme samples (10 mg) were radiolabelled by the attachment of ¹²⁵I using Pierce IODO-GEN® Pre-Coated Iodination Tubes. The borosilicate tubes are coated with the iodination reagent (1,3,4,6-tetrachloro-3α-6α-diphenylglycouril) and supplied ready for use. ¹²⁵I was purchased from Amersham, and the Direct Method procedure as detailed by Pierce followed. Excess reagent was removed by passing samples though Sephadex PD10 desalting columns.

Radioactivity in each sample was determined at this stage, and final dose calculated.

In vivo testing in male Wistar rats showed enzyme activity in liver.

### Example H

1 Introduction

The identification, investigation and implementation of significantly improved protein purification methods to our system, which allows the production of reasonable amounts of pure protein (rhamnosidase activity), have been achieved.

Modifications have been carried out on this pure protein to produce a range of constructs.

Analysis of these constructs has involved gel electrophoresis and enzyme activity. In addition analysis of their stability in solution, to temperature (hot and cold) and to protease enzymes, has been assessed.

The in vivo assessment work has involved the use of gamma scintigraphy to allow visualisation of the drug in the body. In vivo assessment at the cellular level has involved a micro autoradiography study coupled with confocal microscopy. Both allow an assessment of cell-specific delivery of the protein construct to particular cell types within the liver. In addition, confocal microscopy using a rhamnopyranosidic fluorophore has allowed an assessment of prodrug mimic activation.

Stability testing has involved the use of a rat tritosomal preparation^{xxi} to assess the potential stabilities of both protein construct and prodrug in liver lysosomes.

2 Methods & Discussion

2.1 Enzyme Purification

2.1.1 Purification of N-WT (Wild-Type Naringinase)

Naringinase from P. decumbens (200 mg) was dissolved in deionised water (20 ml), placed in cellulose ester dialysis tubing (50 kDa MWCO) and dialysed against deionised water for 24 hours (water changes at 1,3,6,18 hours). Water was removed by freeze-drying yielding a white solid. Yield=150 mg.

2.1.2 Preparation of N-DG (Deglycosylated Naringinase)

A solution of N-WT (10 ml, 10 mg/ml in 10 mM pH6.0 orthophosphate buffer) and a solution of EndoH (50 μl, 10 mg/ml solution) were combined in a sample vial fitted with a magnetic stirrer bar and heated at 37° C. for 24 hours. The solution was transferred to a Spectrum DispoDialyser® (50 kDa MWCO) and dialysed against deionised water for 24 hours, with water changes at 1, 4, 6 and 18 hours. The resultant solution was freeze-dried yielding a white powder. Purity was assessed by gel electrophoresis (SDS PAGE).

2.1.3 Stability Testing

2.1.3.1 Assessment of Stability of N-WT During Dialysis/Freezing/Thawing/Drying

N-WT (80 mg) was dissolved in water (8 ml) and dialysed using a SpectraPor DispoDialyser (50 kDa MWCO) against water for 24 hours. Three 500 μl aliquots were taken from the resultant solution and treated as follows:
1. No further treatment, assessment of composition by 10% SDS PAGE
2. Sample frozen, thawed, assessment of composition by 10% SDS PAGE
3. Sample freeze-dried, assessment of composition by 10% SDS PAGE

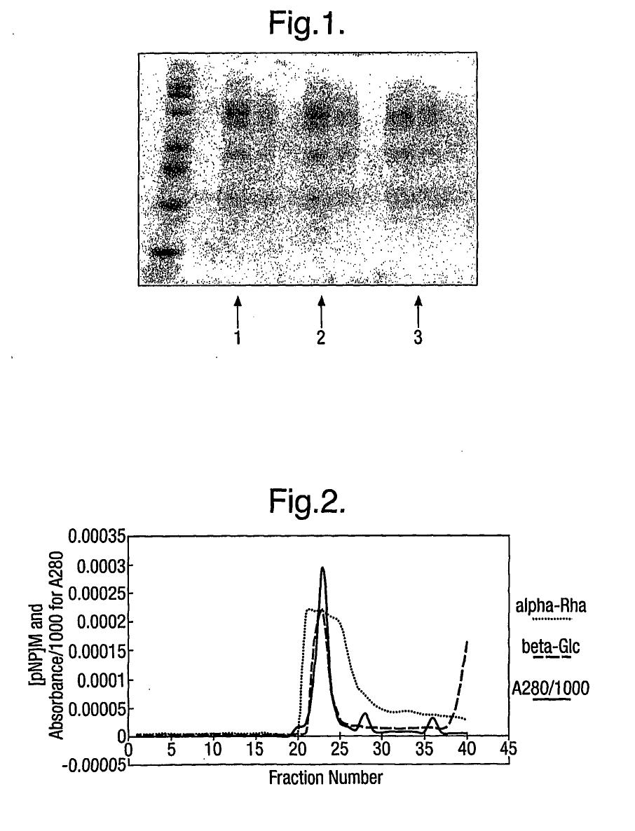

For results see FIG. 1. The left-hand lane in the Figure shows molecular weight markers.

2.1.3.2 Solution Stability of N-WT at Room Temperature

A solution of N-WT (10 ml, 5 mg/ml) in orthophosphate buffer (pH7.0, 0.1M) was stirred slowly in a 50 ml round bottom flask for 14 days at room temperature. Aliquots were taken at the periods shown, diluted to 0.5 mg/ml and a kinetic assay carried out (pH6.8, 7.0, 7.2, with pNP-Rha as substrate).

Sample Times: 1, 2, 3, 5, 7, 9, 14 Days

2.1.3.3 Solution Stability of N-WT at 37° C.

A solution of N-WT (10 ml, 5 mg/ml) in orthophosphate buffer (pH7.0, 0.1M) was stirred slowly in a 50 ml round bottom flask for 14 days at 37° C. Aliquots were taken at the periods shown, diluted to 0.5 mg/ml and a kinetic assay carried out (pH6.8, 7.0, 7.2, with pNP-Rha as substrate).

Sample Times: 1, 2, 3, 5, 7, 9, 14 Days

2.1.3.4 Solution Stability of N-WT in the Presence of SBL

A solution of N-WT (10 ml, 5 mg/ml) in orthophosphate buffer (pH7.0, 0.1M) was stirred slowly in a 50 ml round bottom flask with an aliquot of SBL solution (1 mg, pre-prepared in pH7.0 orthophosphate buffer, freeze dried) for 14 days. Aliquots were taken at the periods shown, diluted to 0.5 mg/ml and a kinetic assay carried out (pH6.8, 7.0, 7.2, with pNP-Rha as substrate).

Sample Times: 1, 2, 3, 5, 7, 9, 14 Days

2.1.4 Size Exclusion Chromatography

2.1.4.1 BioGel™ P-2 Column (4×70 cm) Purification of N-WT

N-WT (50 mg, 10 mg/ml in 0.1M sodium chloride corrected to pH4.8 with hydrochloric acid) was loaded onto to a column of BioGel™ P-2 (4×70 cm) and eluted using 0.1M sodium chloride corrected to pH4.8 with hydrochloric acid. Fractions (5 ml) were collected and analysed by assessing absorbance at 280 nm and for rhamnosidase and glucosidase activity using pNP-Rha and pNP-Glc substrates (100 μl from column plus 100 μl 3.5 mM substrate solutions, incubated at 37° C.). The results are shown in FIG. 2.

Fractions 21-27, 28-37 and 39-40 were combined, freeze-dried, redissolved and passed down a Sephadex PD10 desalting column and applied to 10% SDS PAGE.

2.1.4.2 BioGel™ P-100 Column Purification of N-WT

2.1.4.2.1 3.5×50 cm Column

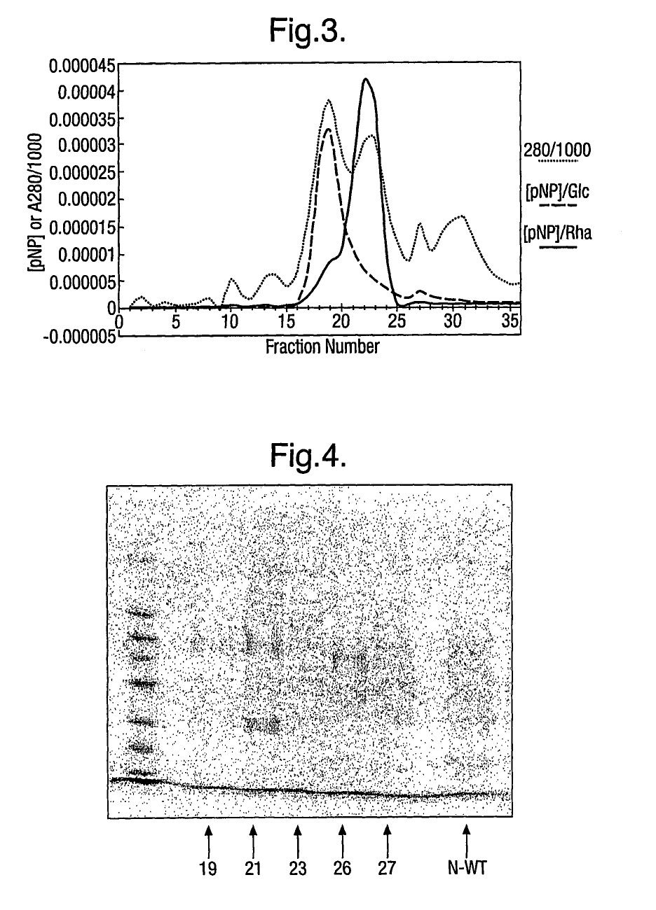

N-WT (50 mg, 8 mg/ml in 0.1M sodium chloride corrected to pH4.8 with hydrochloric acid) was loaded onto to a column of BioGel™ P-100 (3.5×50 cm) and eluted using 0.1M sodium chloride corrected to pH4.8 with hydrochloric acid. Fractions (5 ml) were collected and analysed by assessing absorbance at 280 nm and for rhamnosidase and glucosidase activity using pNP-Rha and pNP-Glc substrates (100 μl from column plus 100 μl 3.5 mM substrate solutions, incubated at 37° C.). The results are shown in FIG. 3.

N.B. For 2.1.4.2.1, the sample of N-WT was dialysed (50 kDa MWCO) against water prior to application to the column.

Fractions 19, 21, 23, 26 and 27 were then passed down a Sephadex PD10™ desalting column and analysed by 10% SDS PAGE (FIG. 4).

2.1.4.2.2 5.5×45 cm Column

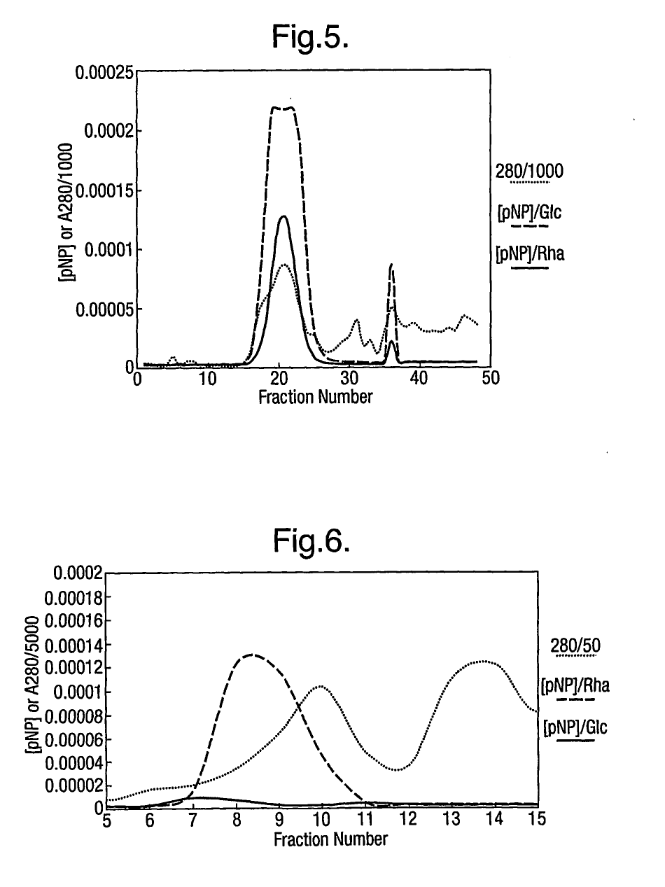

N-WT (150 mg, 10 mg/ml in 0.1M sodium chloride corrected to pH4.8 with hydrochloric acid) was loaded onto to a column of BioGel™ P-100 (5.5×45 cm) and eluted using 0.1M sodium chloride corrected to pH4.8 with hydrochloric acid. Fractions (10 ml) were collected and analysed by assessing absorbance at 280 nm and for rhamnosidase and glucosidase activity using pNP-Rha and pNP-Glc substrates (100 μl from column plus 100 μl 3.5 mM substrate solutions, incubated at 37° C.). The results are shown in FIG. 5.

2.1.4.2.3 5.5×50 cm Column

Predialysed (see section 2.2) N-WT (150 mg, 10 mg/ml in 0.1M sodium chloride corrected to pH4.8 with hydrochloric acid) was loaded onto to a column of BioGel P-100 ™ (5.5×50.0 cm) and eluted using 0.1M sodium chloride, corrected to pH4.8 with hydrochloric acid. Fractions (15 ml) were collected and analysed by assessing absorbance at 280 nm and for rhamnosidase and glucosidase activity using pNP-Rha and pNP-Glc substrates (100 μl from column plus 100 μl 3.5 mM substrate solutions, incubated at 37° C. for 2 minutes). Appropriate fractions were combined, freeze-dried and desalted using Sephadex G25™ (PD10 column).

2.1.4.2.4 2.5×75 cm Column

Predialysed (see section 2.2) N-WT (150 mg, 10 mg/ml in 0.1M sodium chloride corrected to pH4.8 with hydrochloric acid) was loaded onto to a column of BioGel P-100 ™ (2.5×75.0 cm) and eluted using 0.1M sodium chloride, corrected to pH4.8 with hydrochloric acid. Fractions (12 ml) were collected and analysed by assessing absorbance at 280 nm and for rhamnosidase and glucosidase activity using pNP-Rha and pNP-Glc substrates (100 μl from column plus 100 μl 3.5 mM substrate solutions, incubated at 37° C. for 2 minutes). Appropriate fractions were combined, freeze-dried and desalted using Sephadex G25 (PD10 column).

2.1.4.2.5 3.2×50.0 cm—Millipore Vantage-L™ Column

Predialysed (see section 2.2) N-WT (250 mg, 20 mg/ml in 0.1M sodium chloride corrected to pH4.8 with hydrochloric acid) was loaded onto to a column of BioGel P-100 ™ (3.2×50.0 cm—Millipore Vantage-L™ chromatography column) and eluted using 0.1M sodium chloride, corrected to pH4.8 with hydrochloric acid (flow rate=20 ml/hr, controlled using a peristaltic pump). Fractions (15 ml) were collected and analysed by assessing absorbance at 280 nm and for rhamnosidase and glucosidase activity using pNP-Rha and pNP-Glc substrates (100 μl from column plus 100 μl 3.5 mM substrate solutions). Appropriate fractions were combined, freeze-dried and desalted using Sephadex G25™ (PD10 column). The results are shown in FIG. 6.

2.1.5 Ion Exchange Chromatography

2.1.5.1 Ion Exchange Using Standard Column

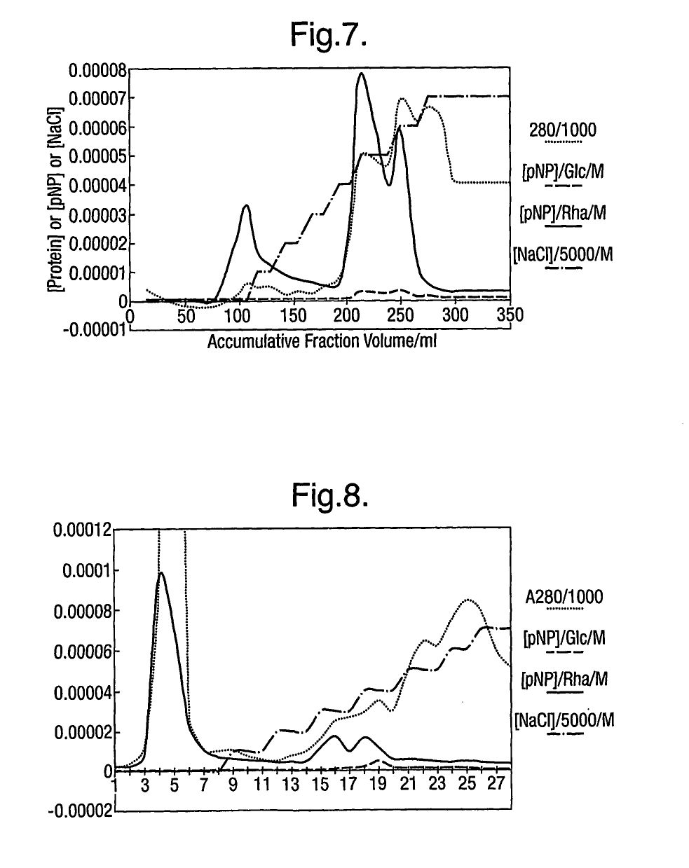

Product of size exclusion purification (2.4 ml, 5 mg/ml in 20 mM L-histidine buffer, corrected to pH6.0) was loaded onto a column of Sepharose™ DEAE (2.0×25.0 cm) and eluted with 20 mM L-histidine buffer, corrected to pH6.0 (100 ml, 15 ml fractions). An increasing salt gradient (0.05-0.35M NaCl, 30 ml of each incremental 0.05M step) was applied and fractions (15 ml) were collected and analysed by assessing absorbance at 280 nm and for rhamnosidase and glucosidase activity using pNP-Rha and pNP-Glc substrates (100 μl from column plus 100 μl 3.5 mM substrate solutions). Appropriate fractions were combined, freeze-dried and desalted using Sephadex G25™ (PD10 column). The results are shown in FIG. 7.

2.1.5.2 Ion Exchange Using Millipore™ Column

Product of size exclusion purification (see section 2.3) (2.4 ml, 5 mg/ml in 20 mM L-histidine buffer, corrected to pH6.0) was loaded onto a column of Sepharose™ DEAE (2.0×25.0 cm—Millipore Vantage-L™ chromatography column) and eluted with 20 mM L-histidine buffer (flow rate=100 ml/hr, controlled using a peristaltic pump), corrected to pH6.0 (100 ml, 15 ml fractions). An increasing salt gradient (0.05-0.35M NaCl, 30 ml of each incremental 0.05M step) was applied and fractions (15 ml) were collected and analysed by assessing absorbance at 280 nm and for rhamnosidase and glucosidase activity using pNP-Rha and pNP-Glc substrates (100 μl from column plus 100 μl 3.5 mM substrate solutions). Appropriate fractions were combined, freeze-dried and desalted using Sephadex™ G25 (PD10 column). The results are shown in FIG. 8.

Summary of Enzyme Purification Work

Initially, yielding a pure band of rhamnosidase activity from crude naringinase (without glucosidase activity or other protein contaminants) was proving difficult, and so this purification was a priority for the project.

Previous purification (prior to glycosylation) had involved dialysis (2.1.1) using cellulose ester tubing with a 50 kDa MWCO. This purifies the naringinase to a certain extent, but still not sufficient for the requirements of a drug delivery system.

One explanation for the constant presence of contaminants was that the naringinase was decomposing during handling so a number of assays were carried out. The work described in section 2.1.3.1 was designed to assess the effect of the standard dialysis/freeze/thawing/drying procedures which are routinely carried out on the samples. Analysis in this case was entirely by SDS PAGE (FIG. 1) that illustrates that these different techniques show no adverse effects to the protein.

Further analysis work was intent on assessing solution stability of naringinase—at room temperature, 37° C. and with a protease (SBL). These assays were designed to allow an assessment of stability to handling (i.e. in solution) but also to investigate potential problems when used as in drug delivery—having a substance that is unstable in solution is unsuitable for drug delivery. Results show that, although some loss of activity is observed, this is not a major problem.

In order to improve the quality of purification, a number of different techniques have been investigated. Size exclusion chromatography using BioGel™ media is the first of those stages. BioGel P-2™ is a resin with a low exclusion limit (2 kDa) and was used as an early comparison. As can be seen (Error! Reference source not found.) little separation of the components of the naringinase mixture is observed—as predicted for a resin with such a low exclusion limit.

BioGel P-100™ is from the same family of resins, but with a much larger exclusion limit (80-100 kDa). This affords a significantly improved level of purification. The gel of fractions form 2.1.4.2.1 (FIG. 4) shows that it is possible to purify bands in particular weight ranges. In particular fraction 23, which showed greatest rhamnosidase activity is significantly different to the wild type sample, in that the higher molecular weight bands are more prominent, suggesting that we have removed much of the lower molecular weight contaminant from the crude mixture.

A number of different columns were than analysed using the same BioGel P-100™ resin. Different widths and heights have been assessed (2.1.4.2.2, 2.1.4.2.3 & 2.1.4.2.4), each having little effect on the quality of separation. A move to using Millipore Vantage-L™ columns allowed the use of a peristaltic pump to pump solvent through the column (previous purifications had been run under gravity or using air pressure). This new column allowed more repeatable purifications to be carried out (3.1.4.2.5), although no significant improvement in separation quality was observed.

Ion exchange chromatography was investigated, using samples from the size exclusion chromatography that were most concentrated in rhamnosidase activity. Initial attempts used a standard chromatography column and pure samples of rhamnosidase activity were produced. The use of a Millipore Vantage-L™ column allowed routine use of this resin to yield significant amounts of pure rhamnosidase activity (see gel in modifications section, FIG. 9).

A purification method is now available which allows 500 mg crude naringinase to yield 30 mg pure rhamnosidase activity, after dialysis, size exclusion chromatography and ion exchange chromatography.

2.2 Enzyme Modification

2.2.1 Preparation of Modification Reagents

2.2.1.1 Synthesis of Gal-TPU Derivative

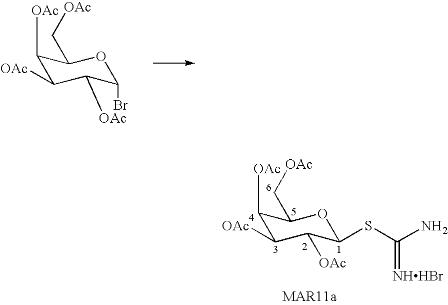

Acetobromo-α-D-galactose (5.0 g, 12.16 mmol) and thiourea (1.2 g, 15.81 mmol) were suspended in dry acetone (50 ml) in a 250 ml round bottom flask fitted with a magnetic stirrer and reflux condenser, under a nitrogen atmosphere. The mixture was refluxed for 2 hours and the reaction followed by tlc, reduced in volume (to 30 ml) in vacuo and chilled for 3 hours. The resulting white crystals (11) were filtered, washed with cold acetone and dried. Yield=4.06 g. Characterisation as described above. 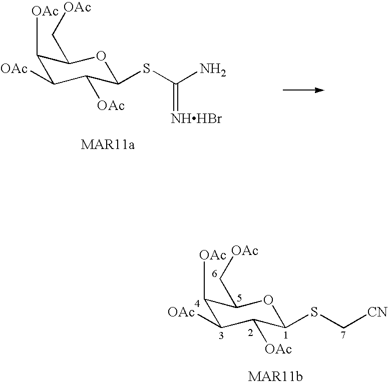
2.2.1.2 Synthesis of Gal-IME Reagent

Gal-TPU (3.7 g, 7.6 mmol) was stirred with dry acetone (20 ml) and water (20 ml) was added. Sodium bisulfite (2.0 g, 38 mmol), potassium carbonate (1.6 g, 11.5 mmol) and chloroacetonitrile (2.5 ml, 40 mmol) were then added in order, and the mixture stirred at room temperature for 3 hours. This mixture was then added to ice water (50 ml) and stirred vigorously for 3 hours at 4° C. Extraction with chloroform (4×40 ml) was performed and the extract washed with sodium chloride solution (2×60 ml, 1M), and the resulting organic layer was dried over anhydrous magnesium sulfate. The organic solution was filtered and all solvent removed in vacuo.

The resulting gum/crystalline material was purified by flash column chromatography (3:1 EtOAc:Hexane) and the appropriate fractions combined and solvent removed in vacuo. Yield=2.05 g, 67%. Characterisation as described above.
2.2.1.3 Synthesis of Per-O-acetomannose

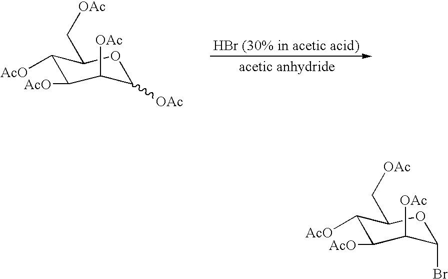

Mannose (4.0 g, 22.2 mmol), pyridine (40 ml) and acetic anhydride (20 ml) were stirred in a 250 ml round bottom flask at room temperature under an inert atmosphere for 24 hours. All solvent was removed in vacuo, HCl (15 ml, 2M) was added, and then product extracted into ethyl acetate (3×20 ml). The combined organic layers were washed with water (3×10 ml), dried over magnesium sulfate and then all solvent removed in vacuo. Characterisation as described above.

This crude product was used immediately in the next stage (2.1.4.2).
2.2.1.4 Synthesis of Acetobromomannose

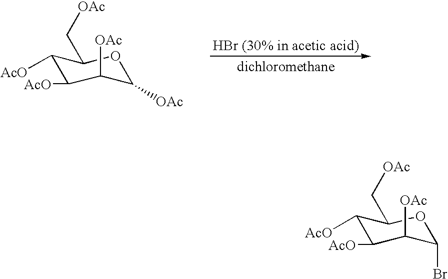

Per-O-acetomannose (8 g, 20.49 mmol), HBr in acetic acid (20 ml, 30%) and acetic anhydride (10 ml) were stirred in a 250 ml round bottom flask under an atmosphere of nitrogen for 24 hours. Additional HBr in acetic acid (5 ml, 30%) was introduced and stirring continued for a further 24 hours. Toluene (30 ml) and acetic anhydride (5 ml) were added, and all solvent removed in vacuo, yielding a dark brown syrup. Flash column chromatography (2:1 EtOAc:Hex) yielded pure product (6.0 g, 13.59 mmol, 66.3%). Characterisation as described above.
2.2.1.5 Synthesis of Man-TPU Derivative

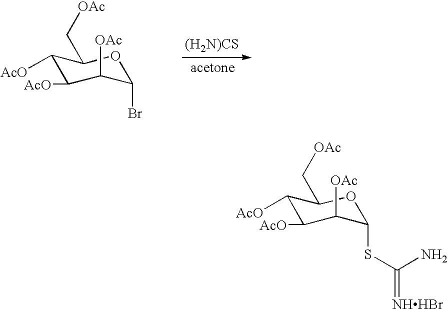

Acetobromomannose (2.677 g, 6.51 mmol) and thiourea (0.655 g, 8.60 mol) were suspended in acetone (25 ml, dry) and refluxed under an inert atmosphere for 3.5 hours. The resultant solution was reduced in volume (˜30%) and chilled to promote crystallisation of product. Crystallisation was achieved after 36 hours, solid was filtered, washed with dry cold acetone and dried in vacuo. Yield=1.3 g, 3.32 mmol (51.0%). Characterisation as described above.
2.2.1.6 Synthesis of Man-IME Derivative

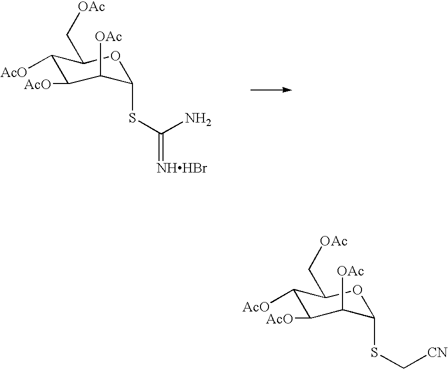

Man-TPU reagent (5.413 g, 13.83 mmol) was dissolved in water (30 ml) and acetone (30 ml), and then sodium bisulfite (2.4 g, 45.6 mmol), potassium carbonate (2.33 g, 16.92 mmol) and chloroacetonitrile (7.2 ml, 115.2 mmol) were added in order. The resultant mixture was stirred for 6 hours. The mixture was poured into ice water (50 ml) and stirred for 1 hour, and a white precipitate formed. Extraction by chloroform (3×80 ml), washing with sodium chloride solution (3×40 ml, 1M) and flash column chromatography (3:1 EtOAc:Hexane) yielded a white solid. Yield=3.539, 8.81 mmol (63.70%). Characterisation as described above.
2.2.1.7 Synthesis of N-Benzyl-4-cyanomethylsulfanyl-butyramide

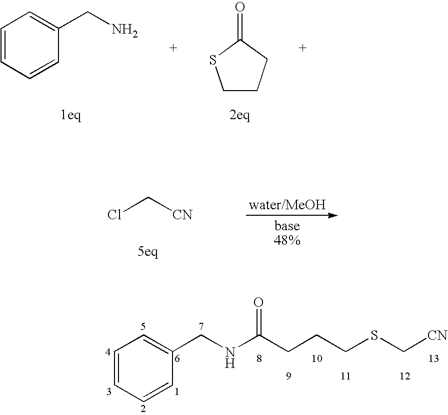

Benzylamine (0.55 ml, 5.0 mmol), γ-thiobutyrolactone (0.87 ml, 10.0 mmol) and chloroacetonitrile (1.58 ml, 25 mmol) were added to an aqueous solution of sodium hydrogen carbonate (30 ml, 0.5M) and methanol (25 ml) in a round bottom flask fitted with reflux condenser, magnetic stirrer bar and inert atmosphere. The mixture was heated overnight at 50° C.

The methanol was removed in vacuo, and the resultant aqueous layer extracted with chloroform (3×60 ml). The organic layers were combined and washed with hydrochloric acid (3×60 ml, 1M) then dried over magnesium sulfate. This solution was filtered, and all solution removed in vacuo.

The product oil was purified by flash chromatography (3:1 EtOAc:Hexane+1% Et₃N).

Yield=0.57 g, 46%. ¹H-NMR (250 MHz CDCl₃) 2.04 (quin, 2H, H¹⁰), 2.36, 2.80 (2×t, 2×2H, H⁹ & H¹⁰), 3.28 (s, 2H, H¹²), 4.43 (d, 2H, H⁷), 5.85 (s, 1H, NH), 7.26-7.31 (5×s, 5H, Ph).
2.2.1.8 Synthesis of Glycodendrimer-IME Reagent

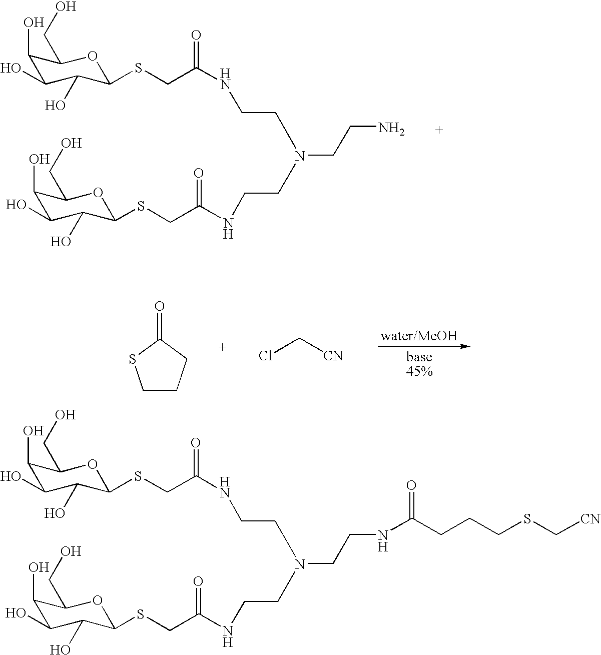

Bis{N-[2-(1-thio-β-D-galactopyranosyl)ethanoyl]aminoethyl}amine (98.9 mg, 0.16 mmol), γ-thiobutyrolactone (0.14 ml, 1.6 mmol) and chloroacetonitrile (0.20 ml, 3.2 mmol) were added to an aqueous solution of sodium hydrogen carbonate (1 ml, 0.5M) and methanol (1 ml) in a round bottom flask fitted with reflux condenser, magnetic stirrer bar and inert atmosphere. The mixture was heated for 24 hours at 50° C.

The mixture was neutralized with 2M HCl, concentrated in vacuo and purified by flash chromatography (CHCl₃: MeOH:H₂O:Et₃N 60:35:7:1) to yield a product, which was taken to dryness in vacuo. The product was then loaded onto Dowex 50W2-200 (H⁺) in water:methanol 1:1 and eluted with 10% NH₃ solution. The product dried by freeze-drying.

Yield=54.7 mg, 45%. ¹H NMR (300 MHz, CD₃OD) ˜1.25 (t, 4H, CH₂CN), 2.18 (q, 2H), 2.5 (t, 2H), 2.7 (t, 4H), 2.9 (dd, 2H), 3.35 (m, 24H), 4.47 (d, 2H, anomeric H). Mass confirmed by ES-MS.

2.2.1.9 Scale Up of 2.2.1.8

Repeat of 2.2.1.8 but on 300 mg scale. Same percentage yield achieved.
2.2.1.10 Synthesis of Peraceto-O-lactopyranose

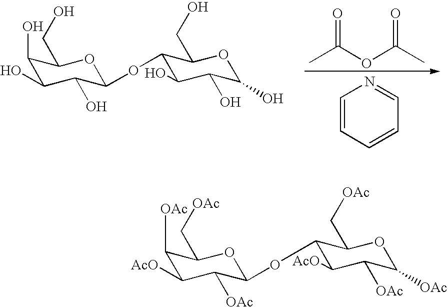

Lactose monohydrate (16.0 g, 44.4 mmol) was suspended in acetic anhydride (64 ml, 678.4 mmol) and pyridine (128 ml, 1,. 56 mol). The mixture was stirred for 24 hours at room temperature under an inert atmosphere. All solvent was removed in vacuo yielding an orange oil which washed with hydrochloric acid (50 ml, 2M) and extracted with ethyl acetate (4×70 ml). Organic layers were combined and washed with water (3×30 ml) then dried over anhydrous magnesium sulfate. All solvent was removed in vacuo yielding a pale yellow gum.

Yield=35.5 g, 50.1 mmol=113%

This product was used crude in the next step, 2.2.1.11.
2.2.1.11 Synthesis of Acetobromolactopyranose

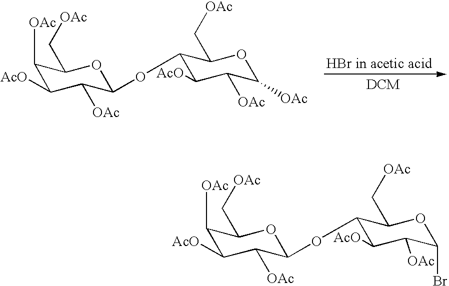

Peraceto-O-lactopyranose (30.9 g, 44.4 mmol) was dissolved in dry dichloromethane (200 ml) and hydrogen bromide in acetic acid (60 ml, 30%) was added dropwise. After 3 hours the solution was poured over ice water, then organic and aqueous layers separated. The organic layer washed with a saturated solution of sodium hydrogen carbonate (3×50 ml), dried over anhydrous magnesium sulfate, filtered and all solvent removed in vacuo yielding a brown oil. Flash column chromatography (2:1 ethyl acetate:hexane) was unsuccessful.

2.2.2 Modification Reactions

2.2.2.1 Preparation of N-WT→GalIME

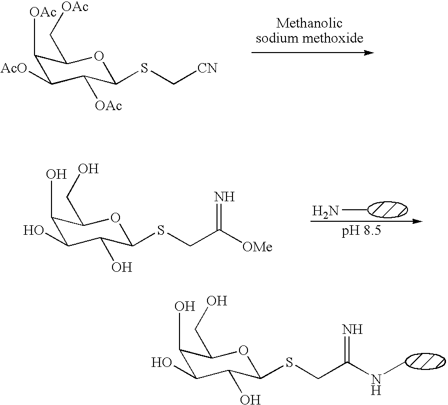

Cyanomethyl Per-O-acetyl-1-thiogalactopyranoside (270 mg, 0.67 mmol) was dissolved in dry methanol (24 ml) in a 50 ml r.b.f. fitted with a magnetic stirrer under an inert atmosphere. A methanolic solution of sodium methoxide (240 μl, 1M) was added and stirring continued at room temperature for 36 hours. All solvent was removed in vacuo yielding a white solid.

N-WT (50 mg) was dissolved in an aqueous solution of sodium tetraborate (0.25M, pH8.5) and added to the white solid and stirred at room temperature for 24 hours.

The solution was then dialysed against deionised water for 12 hours (with water changes at 1, 4, and 6 hours) using Viskin™ dialysis tubing (12-14 kDa), and then passed down a Sephadex G25 PD10™ column. The resultant solution was freeze-dried yielding a white powder.
2.2.2.2 Preparation of N-WT→ManIME

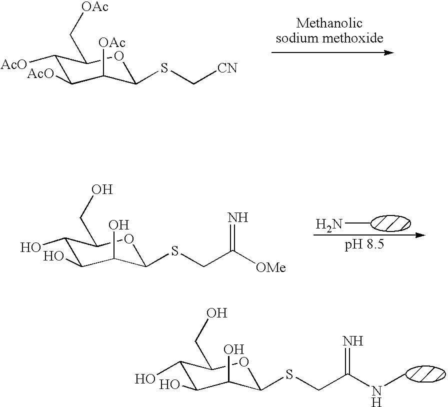

Cyanomethyl Per-O-acetyl-1-thiomannopyranoside (225 mg, 0.56 mmol) was dissolved in dry methanol (20 ml) in a 50 ml r.b.f. fitted with a magnetic stirrer under an inert atmosphere. A methanolic solution of sodium methoxide (200 μl, 1M) was added and stirring continued at room temperature for 36 hours. All solvent was removed in vacuo yielding a white solid.

N-WT (40 mg) was dissolved in an aqueous solution of sodium tetraborate (0.25M, pH8.5) and added to the white solid and stirred at room temperature for 24 hours.

The solution was then dialysed against deionised water for 12 hours (with water changes at 1, 4, and 6 hours) using Viskin™ dialysis tubing (12-14 kDa), and then passed down a Sephadex G25 PD10™ column. The resultant solution was freeze-dried yielding a white powder.
2.2.2.3 Preparation of N-WT→dGalIME

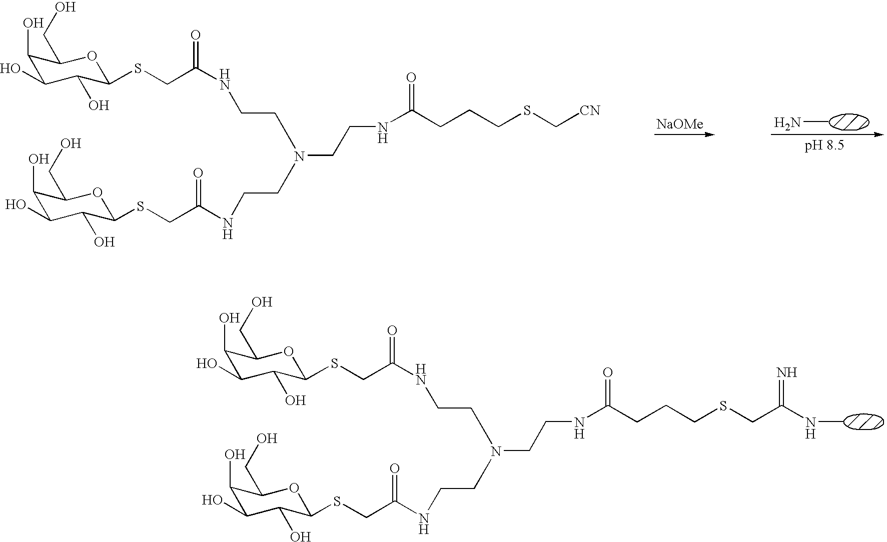

Cyanomethyl dendriGal (510 mg, 0.67 mmol) was dissolved in dry methanol (37 ml) in a 100 ml r.b.f. fitted with a magnetic stirrer under an inert atmosphere. A methanolic solution of sodium methoxide (375 μl, 1M) was added and stirring continued at room temperature for 36 hours. All solvent was removed in vacuo yielding a white solid.

N-WT (50 mg) was dissolved in an aqueous solution of sodium tetraborate (0.25M, pH8.5) and added to the white solid and stirred at room temperature for 24 hours.

The solution was then dialysed against deionised water for 12 hours (with water changes at 1, 4, and 6 hours) using Viskin™ dialysis tubing (12-14 kDa), and then passed down a Sephadex G25 PD10™ column. The resultant solution was freeze-dried yielding a white powder.
2.2.2.4 Preparation of N-DG→GalIME

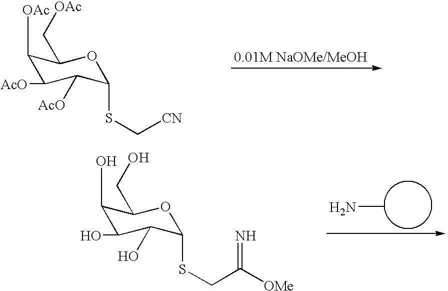

GalIME reagent (155 mg, 0.38 mmol) was dissolved in dry methanol (14 ml) in a 100 ml round bottom flask fitted with a magnetic stirrer under an inert atmosphere. A methanolic solution of sodium methoxide (28 ml, 0.01M) was added, and the mixture stirred for 36 hours at room temperature.

The contents of the flask were taken to dryness in vacuo, and a solution of N-DG (35 mg, 10 mg/ml in 0.25M sodium borate, pH8.5 corrected with cHCl) was added. The mixture was stirred under a nitrogen atmosphere for 24 hours. After this time, the solution was purified by Biogel P-2™ column (2×60 cm, 0.1M NaCl, pH4.8) with presence of protein detected by spectroscopy (absorbance at 280 nm). Appropriate fractions were combined and freeze dried, resulting in a white powder. Purity was assessed by gel electrophoresis (FIG. 9).

1-SDS6 marker
2-N-WT
3-N-WT-GalIME
5-N-DG
6-N-DG-GalIME
7-empty
8-N-WT
9-N-WT-dGalIME

Summary of Modifications Described in Example H

The synthesis of modification reagents has continued from the work described in Examples A & B. Large amounts (>2 g) of the ManIME and GalIME reagents have been synthesised and a sufficient amount of the dendriGalIME reagent is available.

Modification reactions have been continued as previously described, but now with pure rhamnosidase activity as opposed to the crude bands previously used. Analysis after modification (FIG. 9) shows the changes in weight achieved and also the purity of the constructs.

2.3 Enzyme Assay

2.3.1 pH Curve of N-WT

An aqueous solution of N-WT (10 μl, 0.5 mg/ml) was tested for catalytic activity against a range of concentrations of p-nitrophenyl α-L-rhamnopyranoside solutions (190 μl, 3.5, 3.0, 2.5, 2.0, 1.5, 1.0, 0.5, 0.25 mM in 0.1M orthophosphate buffer—pH 5.8, 6.0, 6.2, 6.4, 6.6, 6.8, 7.0, 7.2, 7.4, 7.6, 7.8, 8.0).

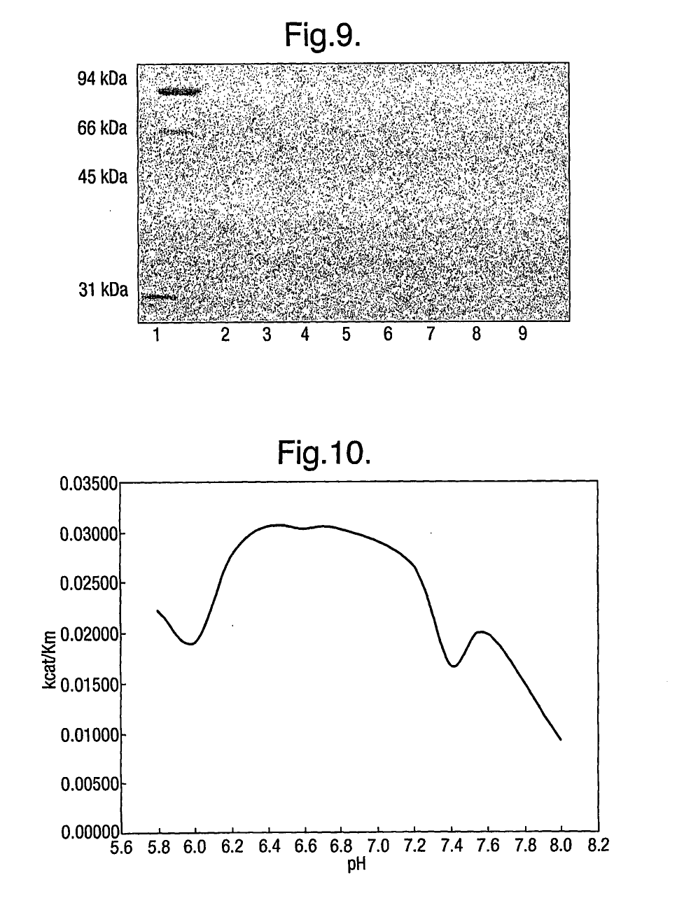

The substrate solution was pipetted into wells on a multiwell plate, and incubated at 37° C. for 5 minutes. The naringinase solution was then added, and the absorbance read at 405 nm for 5 minutes, at 6 second intervals with shaking for 5 seconds before the start of reading, and 1 second before each individual reading. The results are shown in FIG. 10.

2.3.2 pH Curve of N-DG

An aqueous solution of N-DG (10 μl, 5.0 mg/ml) was tested for catalytic activity against a range of concentrations of p-nitrophenyl α-L-rhamnopyranoside solutions (190 μl, 3.5, 3.0, 2.5, 2.0, 1.5, 1.0, 0.5, 0.25 mM in 0.1M orthophosphate buffer—pH 5.8, 6.0, 6.2, 6.4, 6.6, 6.8, 7.0, 7.2, 7.4, 7.6, 7.8, 8.0).

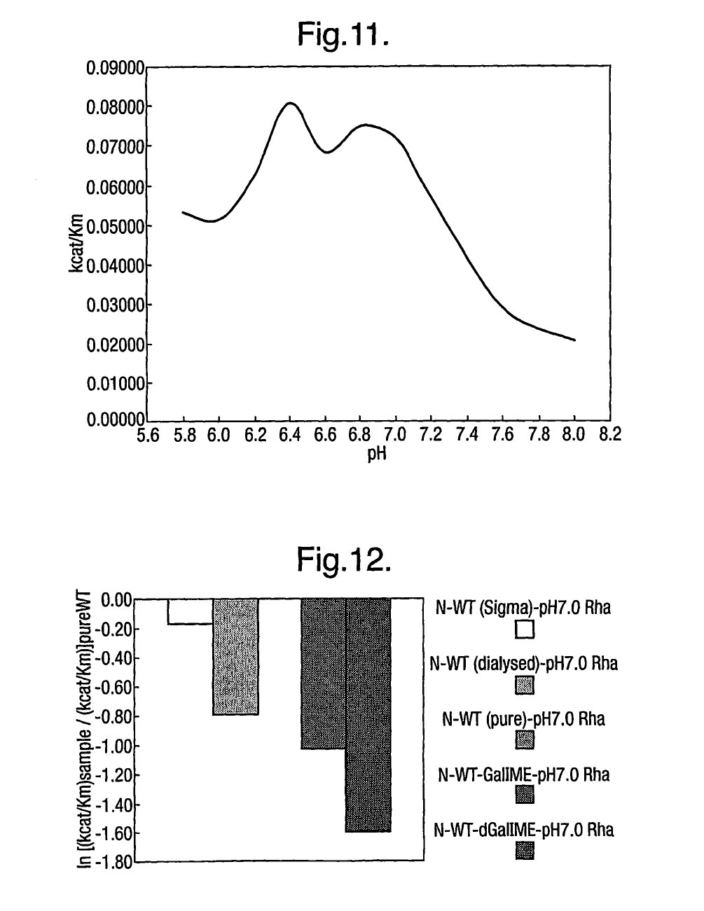

The substrate solution was pipetted into wells on a multiwell plate, and incubated at 37° C. for 5 minutes. The naringinase solution was then added, and the absorbance read at 405 nm for 5 minutes, at 6 second intervals with shaking for 5 seconds before the start of reading, and 1 second before each individual reading. The results are shown in FIG. 11.

2.3.3 Enzyme Assay

An aqueous solution of enzyme sample (10 μL, 0.5 mgml⁻¹) was tested for catalytic activity against a range of concentrations of p-nitrophenyl α-L-rhamnopyranoside solutions (190 μl, 3.5, 3.0, 2.5, 2.0, 1.5, 1.0, 0.5, 0.25 mM in 0.2M orthophosphate buffer—pH 6.8, 7.0, 7.2) and also range of concentrations of p-nitrophenyl β-D-glucopyranoside solutions (190 μl, 3.5, 3.0, 2.5, 2.0, 1.5, 1.0, 0.5, 0.25 mM in 0.2M orthophosphate buffer—pH 6.8, 7.0, 7.2)

The substrate solution was pipetted into wells on a multiwell plate, and incubated at 37° C. for 5 minutes. The enzyme solution was then added, and the absorbance read at 405 nm for 10 minutes, at 6 second intervals with shaking for 5 seconds before the start of reading, and 1 second before each individual reading.

TABLE 2
Rhamnosidase activities

| | | | | k_{cat}/K | ln |
|---|---|---|---|---|---|
| | Vₘₐₓ/ | | k_{cat}/ | ₘ/s₋₁ | [(k_{cat}/Kₘ₎ₛₐₘₚₗₑ/ |
| | Ms⁻¹ | Kₘ/M | s⁻¹ | M₋₁ | (k_{cat}/Kₘ)]pure WT |
| N-WT (Sigma)- | 4.10E− | 1.84E− | 0.13 | 71.2 | −0.17 |
| pH 7.0 Rha | 08 | 03 | | | |
| N-WT (dialysed)- | 3.22E− | 1.62E− | 0.62 | 38.2 | −0.79 |
| pH 7.0 Rha | 07 | 02 | | | |
| N-WT (pure)- | 1.71E− | 3.37E− | 2.84 | 84.2 | 0.00 |
| pH 7.0 Rha | 06 | 02 | | | |
| N-WT-GalIME- | 6.83E− | 3.77E− | 1.14 | 30.1 | −1.03 |
| pH 7.0 Rha | 07 | 02 | | | |
| N-WT-dGalIME- | 2.37E− | 2.32E− | 0.39 | 17.0 | −1.60 |
| pH 7.0 Rha | 07 | 02 | | | |

Summary of enzyme assays

Apart from the standard assays carried out as part of purifications and stability assessments, an investigation into the effect of pH and also assays on some of the new pure constructs have been undertaken. The pH curves show results that could be predicted—as previously shown, a pH optimum at just below 7.0, with decreased activity either side of that optimum.

Table 2 Illustrates the effect of purification and modification on the rhamnosidase activity of the samples. See also FIG. 12.

Glucosidase activity (trace—not able to fit data) was detected in the first two samples—crude preparation from Sigma and after dialysis—but not after further purification (i.e. after ion exchange chromatography).

2.4 Poly-L-lysine (PLL) Modification

2.4.1 Mass Spectral Analysis of PLL Samples

a) PLL-HBr (˜24 kDa) was dissolved in deionised water (2 mg/ml) and diluted by a factor of one sixth with 1:1 water:acetonitrile (+0.5% formic acid).

Samples were analysed on a MicroMass LCT™ via syringe pump introduction.

b) PLL-HBr (˜24 kDa) was dissolved in deionised water (2 mg/ml) and desalted using a Sephadex PD10™ column. The appropriate fractions (ninhydrin detection) were collected and combined and diluted by a factor of one sixth with 1:1 water:acetonitrile (+0.5% formic acid). Samples were analysed on a MicroMass LCT™ via syringe pump introduction.

c) PLL-HBr (˜24 kDa) was dissolved in deionised water (50 mg, 10 mg/ml) and dialysed against water using a DispoDialyser™ (2 kDa MWCO). The resulting solution was freeze dried. Desalted samples were dissolved in deionised water (2 mg/ml) and diluted by a factor of one sixth with 1:1 water:acetonitrile (+0.5% formic acid). Samples were analysed on a MicroMass LCT™ via syringe pump introduction.
2.4.2 Mannose Modification of Poly-L-lysine—500:1

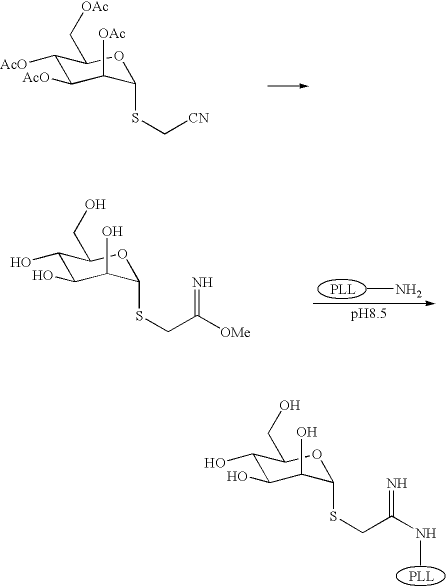

Mannose-IME reagent (266 mg, 0.66 mmol) in dry methanol (60 ml) was stirred under a nitrogen atmosphere with methanolic sodium methoxide (4.5 ml, 0.1M) for 24 hours. All solvent was removed in vacuo.

Poly-L-lysine hydrobromide (50 mg, MW≈24 kDa) was dissolved in sodium tetraborate solution (5 ml, 0.25M, pH8.5), added to the residue from the first stage and stirred at room temperature for 36 hours. An off-white precipitate formed, and so further sodium tetraborate solution (75 ml, 0.25M, pH8.5) was added to aid dissolution before dialysis.

The solution was then dialysed using SpectraPor Cellulose Ester™ dialysis tubing (12-14 kDa MWCO) for 24 hours, against deionised water, and then freeze-dried yielding a white powder.
2.4.3 Mannose Modification of Poly-L-lysine—100:1

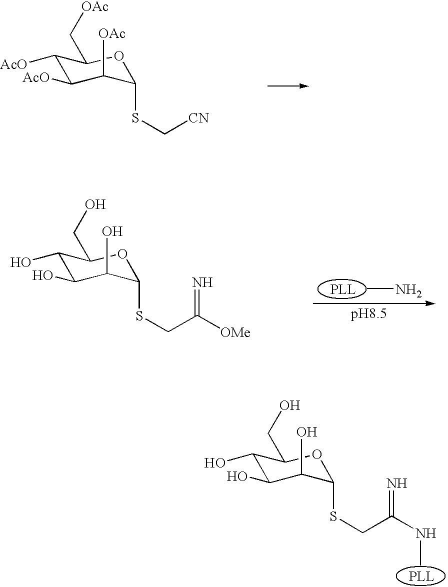

Mannose-IME reagent (53 mg, 0.13 mmol) in dry methanol (60 ml) was stirred under a nitrogen atmosphere with methanolic sodium methoxide (4.5 ml, 0.1M) for 24 hours. All solvent was removed in vacuo.

Poly-L-lysine hydrobromide (50 mg, MW≈24 kDa) was dissolved in sodium tetraborate solution (5 ml, 0.25M, pH8.5), added to the residue from the first stage and stirred at room temperature for 24 hours.

The solution was then dialysed using a SpectraPor DispoDialyser™ (2 kDa MWCO) for 24 hours, against deionised water, and then freeze-dried yielding a white powder.

2.4.4 NMR Analysis of Mannose Modifications of Poly-L-lysine

The products from experiments 2.4.1 and 2.4.2 were analysed using proton NMR. 20 mg of each sample was dissolved in 0.7 ml of deuterated H₂O and spectra were acquired on a 250 MHz machine. As a reference, non-modified poly-L-lysine was prepared as a sample in a similar way to the modified samples and a spectrum acquired.

2.4.5 SDS PAGE of Poly-L-lysine Samples

Samples of poly-L-lysine, prepared in the standard way for gel electrophoresis, were applied to a 10% SDS PAGE system.

No sample was detected on the gel after staining.

2.5 In Vivo Work—Gamma Camera Imaging

A number of radiolabelled constructs (see below) were dosed to New Zealand White rabbits (approximately 1 kg) and imaged using gamma scintigraphy to investigate in vivo distribution.

TABLE 3
Summary of experiment - doses

| Set | Dose | Blocker? | Number of animals |
|---|---|---|---|
| 1 | N-WT | x | 4 |
| 2 | N-WT→GalIME | x | 4 |
| 3 | N-WT→GalIME | ✓ - a | 4 |
| 4 | N-WT→ManIME | x | 4 |
| 5 | N-WT→ManIME | ✓ - b | 4 |
| 6 | N-WT→dendriGalIME | x | 4 |
| 7 | N-WT→dendriGalIME | ✓ - a | 4 |
| | | Total = | 28 |

a - asialofetuin (100 mg/kg)
b - mannosylated polylsine (100 mg/kg)

1. Animals were sedated with Hypnorm (intramuscular or subcutaneous).
2. If appropriate, blocker (100 mg/kg) was dosed intravenously 10 minutes prior to protein dose.
3. ¹²³I-labelled^{xxii} protein (2.5 mg/kg in 1 ml or less of PBS) was dosed intravenously. Total maximum intravenous liquid dose was 2.0 ml/kg.
4. A gamma camera was used to image drug distribution over time (0, 10, 30, 60, 90 and 120 minutes post-dose).
5. Blood samples were taken from the ear vein (10, 30, 60 and 120 minutes post-dose). Sample volumes were in accordance with LASA guidelines (max 4 ml/kg).
6. At the end of the study, the animals were sacrificed and drug distribution confirmed via dissection and organ analysis.

The protein constructs (N-WT, N-WT→GalIME, N-WT→ManIME, N-WT→dGalIME) were labelled with ¹²³I using the IODO-GEN technique (generation of iodonium ion from solution of Na¹²³I with IODO-GEN reagent allowing electrophilic addition to ortho position of tyrosine rings in protein). ¹²³I is a gamma emitter with an energy level window appropriate for detection using gamma scintigraphy. This technique allows an assessment of in vivo distribution of the protein construct over a period of time (2 hours). In addition, standard dissection/organ distribution studies were used to supplement data from the gamma scintigraphy.

The three modified constructs were each dosed in two sets—with and without is a blocker of their particular uptake mechanism. The galactosylated constructs were blocked with asialofetuin (AF), a known blocker of the asialoglycoprotein receptor (ASGPR) and mannosylated constructs were blocked with mannosylated polylysine, both at doses some forty times higher than the construct dose.

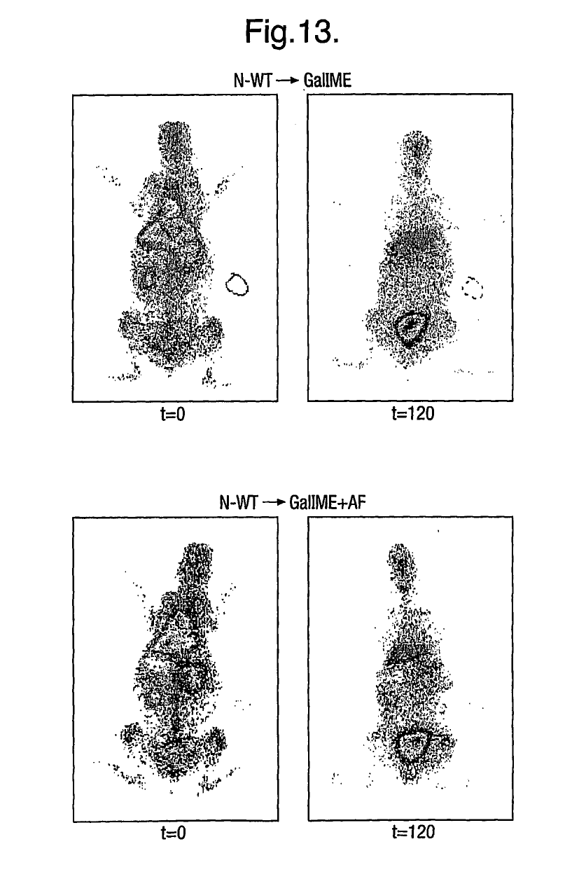

FIG. 13 allows a visual analysis of the effect of blocking uptake of galactosylated samples with asialofetuin. By using this blocker in great excess it is possible to see a change in activity in the liver (and therefore hepatocytes) due to a lack of receptor mediated endocytosis (RME) into the hepatocytes when blocker is present.

Each hepatocyte has some 500,000 ASGPR's on its surface and so even when a large excess of blocker is introduced to the animal, this blocking capacity will eventually diminish. (RME is an active process and the cell takes up the AF, it doesn't simply block a particular receptor for a long period of time). Due to the limits of dissolution of the AF and the maximum volume that can be injected into an animal, it is necessary to accept that this blocking action has a finite lifetime. This is borne out in the time curves seen from determining standardised activity in the liver.

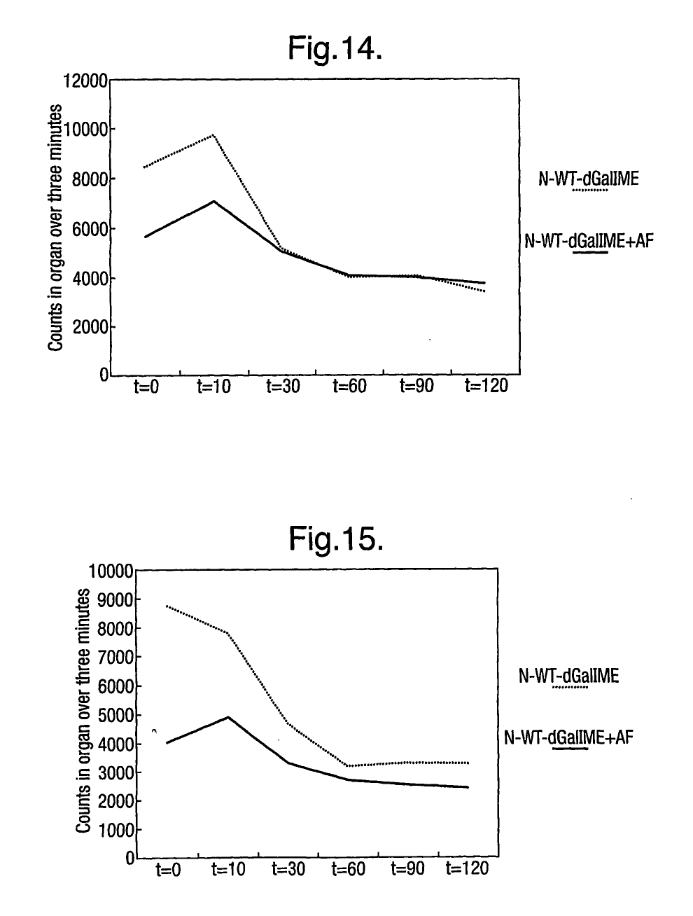

We can also see the liner residence time over the two hour period of the study (FIGS. 14 and 15). Initially, whilst the predosed AF is still present and blocking the RME of the galactosylated sample, a marked difference between the samples with and without AF is observed. Through the course of the study (2 hours) as the AF is removed from circulation and its effects become less pronounced this difference is reduced.

The amount of activity may reduce due to a number of factors:
1. Initially, some of the activity observed is due to construct circulating in the blood.
2. Some construct, on reaching the liver may be metabolised.
3. The gamma camera pictures show large amounts of construct in the bladder after 2 hours. This is due to a large excess of dose been given. This will clearly affect the ability of the body to selectively take up the construct via RME, and hence the presence of the construct in the bladder as it is excreted renally. This may also effect some non-selective uptake in the liver—perhaps into Kupffer cells which would excrete the construct.

The important observation though is that a difference of delivery with/without blocker is observed and also that some significant activity is retained after two hours.

2.7 In Vivo Work—Microautoradiography and Confocal Microscopy

A: Results/Discussion

2.7.1 Assessment of Fluorescence of Tissue Tek OCT™

Tissue Tek OCT is a proprietary product designed for immobilising frozen, tissue samples. The medium allows rapid preservation of samples that can be later processed, for example by taking sections on a cryostat microtome, for use in further studies. As fluorescence was to be used for assessing prodrug activation using sections prepared from the Tissue Tek OCT immobilised samples, it was essential to determine that Tissue Tek OCT would not interfere with these studies. A thin layer of Tissue Tek OCT was sandwiched between two microscope slides and fluorescence determined by excitation at 365 nm. No fluorescence was observed.

In addition, when focussing the confocal microscope (see later) any residual fluorescence in the sample would have been observed before the rhamnopyranosidic fluorophore was added. The only fluorescence detected at this stage was due to the sample itself—many tissues have low levels of endogenous fluorescence.

2.7.2 ³H-Labelling

The use of N-succinimidyl-[2,3-³H] propionate to tritiate proteins is a well-established method.^{xxiii} Tritium is the isotope of choice for this microautoradiography study due to its radiation energy providing high resolution of imaging compared to other isotopes. Although supplied as a solution in toluene, the label was used in aqueous solution (toluene having been removed in vacuo) at pH8.0. It has been documented^{xxiii} that propionation of lysine residues is achievable at a pH range of 3-8. In addition tyrosine residues may be propionated at pH8, and as the naringinase constructs are glycosylated through lysine residues, an alkali pH, 8.0, was chosen to facilitate labelling of tyrosine residues. It should be remembered that higher pH values would lead to hydrolysis of the N-succinimidyl-[2,3-³H] propionate label before protein labelling had occurred. Extended labelling times of up to 24 hours are possible, though in the interests of protein stability, a reaction time of two hours was used.

2.7.3 In Vivo Distribution Study

Microautoradiography is a technique often used to determine cellular localisation of agents within the body. The method described here is based on previous in vivo studies in this project. A protein dose of 2 mg/kg was given to male Wistar rats, with and without a blocking agent, dosed at 100 mg/kg. For the galactose modified constructs this blocker was asialofetuin, a known ligand for the asialoglycoprotein receptor, and for the mannose modified constructs this blocker was PLL-Man. The animals were sacrificed 20 minutes post dose and liver and kidneys removed. The liver samples were processed in two ways. Firstly, two 3 mm slices were taken from separate lobes, immobilised in Tissue Tek OCT™ on a cork ring and frozen in a solvent bath at −40° C. for use in confocal microscopy studies. Secondly, the remaining liver and kidney samples were stored separately in buffered formalin for processing for microautoradiography studies.

2.7.4 Sample Processing for Micro Autoradiography

The liver and kidney samples stored in buffered formalin were processed for microautoradiography by impregnating with and mounting in wax. This process was achieved using a Tissue Tek VIP™ machine. Once set, the wax block could be sliced to produce 4 μm thick tissue sections for analysis. These sections were prepared on a microtome, and floated onto Vectabond-coated microscope slides. Once dried, these slides provide a stable basis for further manipulation. The wax has to be removed for autoradiography, and this is achieved by dipping the slides in xylene baths, and an IMS→water gradient of baths, replacing all organic residues with water. These slides were then dipped in Ilford nuclear emulsion and stored in the dark before processing.

2.7.5 Confocal Microscopy

Fluorescent prodrug mimics have been used to probe the activation of prodrugs by naringinase in the liver. This allows an assessment of the activity of naringinase samples once located in the liver and also of construct localisation. As the fluorescent probe, as with future prodrugs, is a rhamnopyranoside, no mammalian enzymes are available to cleave the probe or prodrug. Therefore any fluorescence observed in the system after the prodrug mimic has been added must be due to activation by the delivered construct (naringinase).

Samples were prepared by taking 7 micron slices of the Tissue Tek OCT-immobilised liver samples on a cryostat microtome. These slices were loaded onto a microscope coverslip and then into a chamber designed to fit the confocal microscope. A buffer designed to mimic physiological conditions was layered over the sample, and the microscope focused on the surface of the sample. Scanning images of the surface and fluorescence images were recorded—no significant fluorescence was observed at this stage. A solution of 4-methylumbelliferyl α-L-rhamnopyranoside (50 μl, 2.0 mM) was added to the chamber and fluorescence detection continued. In the case of N-DG→dGalIME (the only sample assessed to date) fluorescence was detected, and attributed to activation of the prodrug mimic by the construct located in hepatocytes of the liver sample.

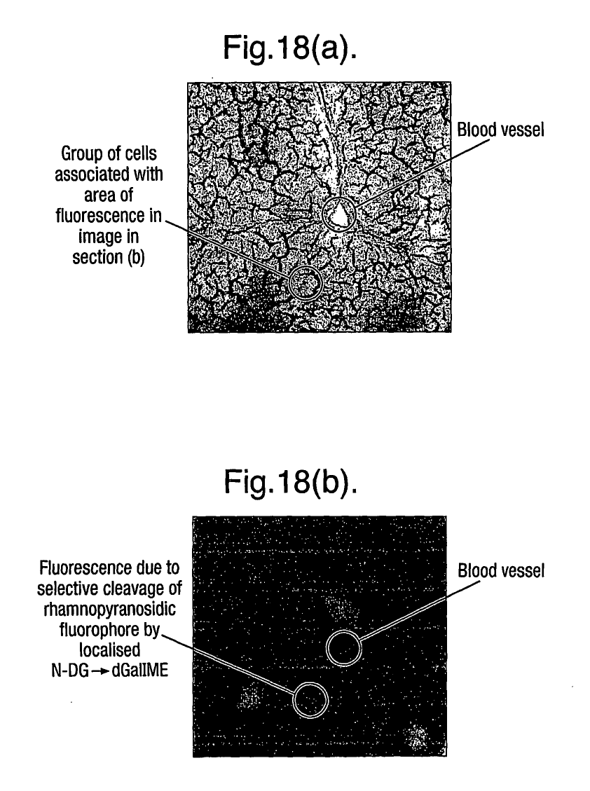

Results are shown in FIG. 18. FIG. 18a shows an image of a section from liver of N-DG→dGalIME dosed animal under phase imaging conditions. FIG. 18b shows the same section in the same orientation under fluorescence imaging conditions with 4-methylumbelliferyl α-L-rhamnopyranoside.

B: Methods

2.7.1 Assessment of Fluorescence of Tissue Tek OCT™

Tissue Tek OCT was layered between two microscope slides and placed in a fluorescence spectrometer, excited at 365 nm and emission recorded at 375-700 nm. As a control, two microscope slides without Tissue Tek OCT was subjected to the same conditions. No fluorescence was detected.
2.7.2 ³H-Labelling

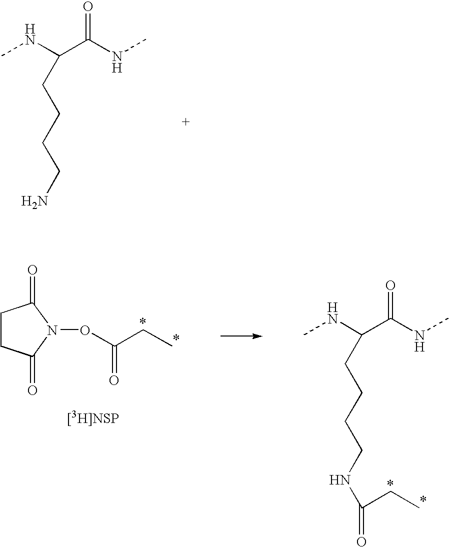

³H-NSP (4.30 mCi, 94.0 Ci/mmol, APBiotech TRK556™, Batch 97, 1 mCi=37 MBq) was supplied in toluene, which was removed in vacuo, and the resultant white solid dissolved in sodium tetraborate solution (6.3 ml, 0.1M pH8.0). This solution was added to solutions of protein in sodium tetraborate buffer as detailed in table below. After two hours reaction time, the protein was purified by size exclusion chromatography using a Sephadex G25 PD10™ column, eluting with phosphate-buffered saline. Radioactivity was determined using scintillation counting.

| | Mass | Vol protein | Vol radiolabel | Radioactivity/ |
|---|---|---|---|---|
| | protein/mg | buffer/ml | buffer/ml | MBq |
| N-WT | 1.5 | 0.20 | 0.45 | 0.32 |
| N-WT→GalIME | 3.0 | 0.40 | 0.90 | 0.39 |
| N-WT→ManIME | 3.0 | 0.40 | 0.90 | 0.26 |
| N-WT→dGalIME | 3.0 | 0.40 | 0.90 | 0.39 |
| N-DG | 1.5 | 0.20 | 0.45 | 0.37 |
| N-DG→GalIME | 3.0 | 0.40 | 0.90 | 0.32 |
| N-DG→ManIME | 3.0 | 0.40 | 0.90 | 0.35 |
| N-DG→dGalIME | 3.0 | 0.40 | 0.90 | 0.31 |

2.7.3 In Vivo Distribution Study

1. Male wistar rats (approx. 250 g) were weighed and tail marked for identification purposes (two animals per dose type).

2. The rats were kept in a hot-box at 38° C. for approximately 15 minutes prior to use to dilate the tail veins.

3. The animals were anesthetized using a solution of 2 ml Hypnorm (fentanyl/fluanisone)+2 ml Hypnovel (midazolam)+10 ml water for injection, via an indwelling needle implanted into the tail vein. 0.2 ml of anaesthetic was administered for induction of anaesthesia, and 0.1 ml was given as a top-up dose as required in order to maintain anaesthesia throughout the study. The rats were positioned ventral side up on a heated table or mat to maintain body temperature (36-40° C.). The rats were surgically prepared by making a midline incision in the neck followed by cannulation of the carotid artery, to allow collection of serial blood samples. 0.25 mg/ml heparin in 0.9% saline was instilled into the carotid cannula to prevent clot formation between samples.

4. If appropriate, blocker (100 mg/kg in PBS) was dosed intravenously via the tail vein 10 minutes prior to protein dose. (Details of dosing in table below)

5. ³H-labelled protein (2 mg/kg, in 1 ml or less of PBS) was dosed intravenously. Total maximum intravenous dose (including test substances and anaesthetic) was 5.0 ml/kg, in accordance with LASA guidelines.

6. Blood samples were taken from a superficial vein or a previously implanted cannula (if applicable) to produce a concentration-time profile of drug in the blood (0, 1, 5, 10, 15 and 20 minutes post-dose). Maximum total blood sample volume was 6.5 ml/kg (equivalent to 10% of total blood in body).

7. At the end of the study, the animals were sacrificed by overdose (Euthatal), and livers and kidneys removed for further analysis. Two 2 mm slices were taken from each liver and frozen in Tissue Tek OCT onto a cork disk, and the remainder stored in buffered formalin.

| | Asialofetuin | PLL-Man | No. of animals |
|---|---|---|---|
| N-WT | x | x | 2 |
| N-WT→GalIME | x | x | 2 |
| | ✓ | x | 2 |
| N-WT→ManIME | x | x | 2 |
| | x | ✓ | 2 |
| N-WT→dGalIME | x | x | 2 |
| | ✓ | x | 2 |
| N-DG | x | x | 2 |
| N-DG→GalIME | x | x | 2 |
| | ✓ | x | 2 |
| N-DG→ManIME | x | x | 2 |
| | x | ✓ | 2 |
| N-DG→dGalIME | x | x | 2 |
| | ✓ | x | 2 |

2.7.4 Sample Processing for Micro Autoradiography

Two 3 mm slices were taken from each liver and kidney samples and mounted into a processing cassette. These cassettes were loaded onto a Tissue Tek VIP™ machine for a standard wash/wax procedure, then suspended in wax and cooled. The surface of the wax blocks was prepared by taking 10 micron thick slices on a microtome to reveal the sample tissue, followed by 4 micron thick slices which were floated onto the surface of Vectabond™ coated microscope slides (10 slides per tissue sample were prepared). After being dried overnight in an oven at 37° C., the residual wax was removed by dipping in xylene and IMS→water gradient tanks. The slides were then coated with Ilford nuclear emulsion (range of particle sizes), dried and stored in the dark.

2.7.5 Confocal Microscopy

Tissue samples stored in Tissue Tek OCT™ were prepared for confocal microscopy by taking 7 micron thick slices on a cryostat microtome and loading on a microscope cover slip. These coverslips were loaded into a confocal microscopy cell and submerged in physiological buffer (500 μl, 145 mM NaCl, 5 mM KCl, 2 mM CaCl₂, 1 mM MgCl₂, 10 mM HEPES, 10 mM glucose, pH7.4). Once the microscope had been focused on the section, images were recorded every 8 seconds (Argon laser—360 nM excitation, 405±40 nm emission), and a solution of 4-methylumbelliferyl α-L-rhamnopyranoside (50 μl, 2 mM) added. Fluorescence was detected for a further 2 minutes. A sample of substrate and buffer was imaged as a control reaction.

2.8 Stability Testing

A: Results and Discussion

2.8.1 Tritosome Purification

A rat liver tritosomal preparation was produced according to known methods. This preparation is useful for assessing the stability of constructs delivered to the hepatocytes. Galactosylated constructs are actively taken up into the cell in an endosome by the endocytic action of the asialoglycoprotein receptor. These endosomes fuse with lysosomes in the hepatocytes, were they will be exposed to lysosomal enzymes. These enzymes are present in the body to break down foreign bodies and waste material, and contain proteases and glycosidases, amongst others. It is essential that the constructs delivered to the hepatocytes are able to survive this degradation in order that they can act on the delivered prodrug. It is hoped that the glycosylation process used in the modification of the constructs will indeed act to stabilise the constructs to these enzymes.

2.8.2 Assessment of Protein Content of Tritosomal Preparation

The Bicinchoninic acid method was used to determine the protein content (and therefore enzyme content) of the tritosomal preparation. By calibrating the sample against a known concentration of BSA, it was shown that the preparation contained protein at a concentration of 0.18 mg/ml.

2.8.4 Assessment of Tritosome Activity

2.8.5 Determining Extinction Coefficient of pNP in Tritosomal Assay Conditions

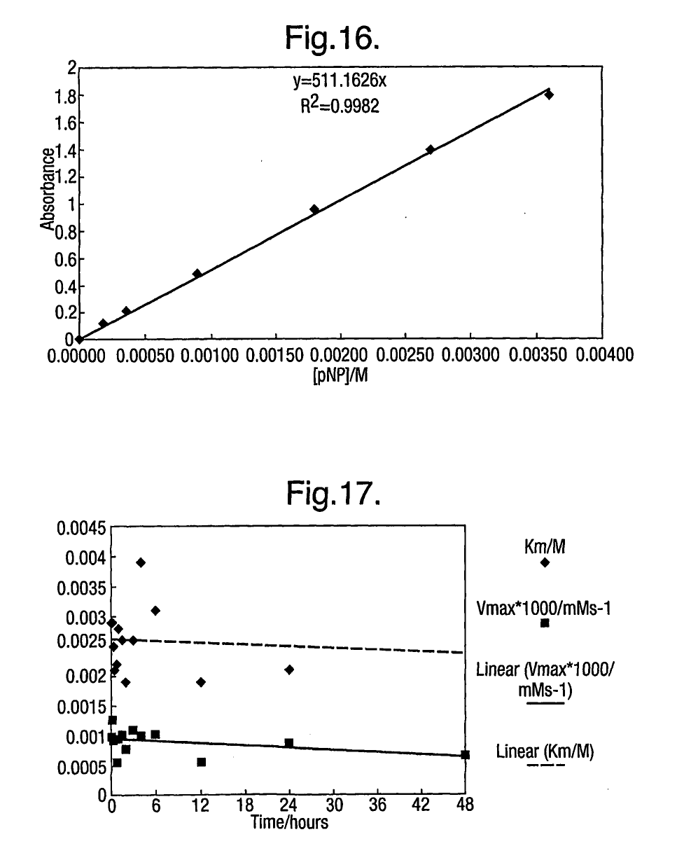

The extinction coefficient of p-nitrophenol varies depending on the conditions, particularly pH, in which it is detected. Previous assays have investigated p-nitrophenol at pH7.0, and so it was necessary to determine the extinction coefficient in the conditions in which the tritosomal stability tests will be carried out. A solution of p-nitrophenol in DMSO was prepared and diluted to gave a concentration gradient in pH5.5 citrate-phosphate buffer with Triton X100, EDTA and GSH. The extinction coefficient of p-nitrophenol under these conditions was found to be 511.2M⁻¹ cm⁻¹ (FIG. 16).

2.8.6 Assessment of Tritosomal Stability of pNP-α-L-Rha (a)

One essential facet of the LEAPT concept is that the rhamnoside-capped prodrugs will be cleaved only by the selectively delivered naringinase construct. To ensure that this is possible, a lysosomal tritosome preparation was used to assess the potential cleavage of α-L-rhamnopyranosides by enzymes in the hepatocytes. p-Nitrophenyl α-L-rhamnopyranoside was used as a chromogenic prodrug mimic. If there are enzymes present in the hepatocytes capable of cleaving α-L-rhamnopyranosidic linkages, this would be detected over an extended assay by the production of p-nitrophenol. Assay conditions based on those used in 2.2.4 were recreated with the used of pNP-α-L-Rha instead of the protease substrate. The mixture was incubated at 37° C. and the absorbance at 410 nm recorded every 5 minutes. No activity was detected after 30 minutes and so a new study (2.8.7) with a higher concentration of pNP-α-L-Rha was designed.

2.8.7 Assessment of Tritosomal Stability of pNP-α-L-Rha (B)

With the concentration of pNP-α-L-Rha increased ten-fold, the tritosomal stability of rhamnopyranosides was reassessed. There was no production of p-nitrophenol after 30 minutes, so the mixture was left at 37° C. for 20 hours. After 20 hours, there was no increase in the level of absorbance at 410 nm, so we can conclude that α-L-rhamnopyranosides are stable in hepatic lysosomes.

2.8.8 Assessment of Tritosomal Stability of pNP-β-D-Gal

Further assessments of the glycosidic enzymes present in the tritosomal preparation were required to allow further design of prodrugs. An assay equivalent to 2.8.7 was used to assess β-D-galactopyranosidic activity. A slight increase in absorbance at 410 nm was detected after 30 minutes and so the mixture was left for 20 hours at 37° C. After this time a significant amount of p-nitrophenol had been released proving β-D-galactopyranosidic activity.

2.8.9 Assessment of Tritosomal Stability of pNP-β-D-Glc

Further assessments of the glycosidic enzymes present in the tritosomal preparation were required to allow further design of prodrugs. An assay equivalent to 2.3.7 was used to assess β-D-glucopyranosidic activity. A slight increase in absorbance at 410 nm was detected after 30 minutes and so the mixture was left for 20 hours at 37° C. After this time a significant amount of p-nitrophenol had been released proving β-D-glucopyranosidic activity.

2.8.10 Determining Extinction Coefficient of pNP in pH5.5 Citrate-phosphate Buffer

The extinction coefficient of p-nitrophenol varies depending on the conditions, particularly pH, in which it is detected. Previous assays have investigated p-nitrophenol at pH7.0, and so it was necessary to determine the extinction coefficient in the conditions in which the long-term construct stability tests will be carried out. A stock solution of p-nitrophenol (0.2M) in citrate-phosphate buffer (0.2M, pH5.5) was diluted to a concentration series and incubated in a multiwell plate (200 μl) at 37° C. The absorbance at 405 nm determined and extinction coefficient calculated to be 521.1M⁻¹ cm⁻¹.

2.8.11 Determining Standard Enzyme Kinetics of N-WT in pH5.5 Citrate-Phosphate Buffer

A standard enzyme assay of N-WT was carried out, but with a change of buffer conditions to pH5.5 0.2M citrate-phosphate buffer, with and without Triton X100 (used as a detergent to disrupt the lysosomal lipid compartment).

| | Vₘₐₓ/Ms⁻¹ | Kₘ/M | k_{cat}/s⁻¹ |
|---|---|---|---|
| MAR317 + TritonX100 | 1.031E−05 | 4.500E−03 | 1.71 |
| MAR317 − TritonX100 | 1.203E−05 | 3.433E−03 | 2.00 |

These results are comparable to previous assays involving N-WT, with a slight reduction in k_{cat} easily attributed to the acidic conditions.

2.8.12 Assessment of Tritosomal Stability of N-WT

N-WT was incubated with tritosome preparation to assess potential stability in hepatic lysosomes. An aliquot was removed at set time points (up to 48 hours) and assessed using a standard assay with pNP-α-L-Rha at pH5.5. The chart in FIG. 17 below illustrates the fact that there is little change in kinetic parameters in time, indicating that naringinase is a good candidate for use in LEAPT.

B: Methods

2.8.1 Tritosome Purification

Rat liver tritosomes were prepared by a standard procedure.^{xxiv}

2.8.2 Assessment of Protein Content of Tritosomal Preparation

The protein content of the tritosome preparation was determined using the Bicinchoninic Acid assay.^{xxv} The BSA calibration chart allowed the concentration of protein in the tritosome preparation to be determined as 0.18 mg/ml.

2.8.3 Assessment of Bound Nap in Stock Solution

A stock solution (1.5 ml, pH5.5 citrate phosphate buffer plus 0.2% Triton X100 and 50 mM reduced glutathione and 10 mM EDTA) and DMSO (5 μ) were mixed in a cuvette and used to blank the spectrometer at 315 nm. A solution of BzPheValArgNap (5 μl, 7.0 mg/ml, 1.09×10⁻⁵ mol/ml) in DMSO was then added to stock solution (1.5 ml) in a separate cuvette and the absorbance at 315 nm determined.

2.8.4 Assessment of Tritosome Activity

A stock solution was prepared (pH5.5 citrate phosphate buffer plus 0.2% Triton X100 and 50 mM reduced glutathione (GSH) and 10 mM EDTA) and used as a basis to prepare EDTA (10 mM) and reduced glutathione (50 mM) solutions. Three cuvettes (one blank and two assays, as in table below) were used to determine the tritosome activity by reading absorbance at 410 nm (substrate solution is BzPheValArgNap in DMSO 7.0 mg/ml, 1.09×10⁻⁵ mol/ml) in a chamber incubated at 37° C.

a) Cuvette Setup

| | Blank - volume/μl | Assay - volume/μl |
|---|---|---|
| 10 mM EDTA | 100 | 100 |
| 50 mM GSH | 100 | 100 |
| Substrate solution | 30 | 30 |
| Stock solution | 740 | 740 |
| Tritosomes | 0 | 30 |
| DMSO | 30 | 0 |

b) Readings from Spectrometer

| Time/min | Blank | Assay 1 | Assay 2 |
|---|---|---|---|
| Before addition | 0.118 | 0.122 | 0.115 |
| 0 | 0.119 | 0.169 | 0.168 |
| 5 | 0.119 | 0.193 | 0.199 |
| 10 | 0.119 | 0.263 | 0.272 |
| 15 | 0.120 | 0.355 | 0.366 |
| 20 | 0.120 | 0.441 | 0.453 |
| 25 | 0.120 | 0.544 | 0.532 |
| 30 | 0.120 | 0.667 | 0.670 |

2.8.5 Determining Extinction Coefficient of pNP in Tritosomal Assay Conditions

A stock solution of p-nitrophenol in DMSO (120 mM, 16.69 mg/ml) was prepared and a concentration gradient prepared as detailed below. Absorbance was read at 410 nm in a chamber incubated at 37° C.

a) Cuvette Setup

| | Volume/μl |
|---|---|
| 10 mM EDTA | 100 |
| 50 mM GSH | 100 |
| 120 mM p-nitrophenol | As below |
| Stock solution | 740 |
| DMSO | As below |

b)

| | Vol. | Vol. | Actual | |
|---|---|---|---|---|
| | pNP/μl | DMSO/μl | [pNP]/mM | Absorbance |
| 1 | 30 | 0 | 3.6 | 1.906 |
| 2 | 22.5 | 7.5 | 2.7 | 1.506 |
| 3 | 15 | 15 | 1.8 | 1.067 |
| 4 | 7.5 | 22.5 | 0.9 | 0.592 |
| 5 | 3 | 27 | 0.36 | 0.314 |
| 6 | 1.5 | 28.5 | 0.18 | 0.223 |
| 7 | 0 | 30 | 0.0 | 0.108 |

2.8.6 Assessment of Tritosomal Stability of pNP-α-L-Rha (a)

A stock solution of p-nitrophenyl α-L-rhamnopyranoside in DMSO (3.1 mg/ml, equivalent to BzPheValArgNap solution) was used in an assay equivalent to 2.8.4.

a) Cuvette Setup

| | Blank - volume/μl | Assay - volume/μl |
|---|---|---|
| 10 mM EDTA | 100 | 100 |
| 50 mM GSH | 100 | 100 |
| Substrate solution | 30 | 30 |
| Stock solution | 740 | 740 |
| Tritosomes | 0 | 30 |
| DMSO | 30 | 0 |

b) Readings from Spectrometer

| Time/min | Blank | Assay 1 | Assay 2 |
|---|---|---|---|
| Before addition | 0.112 | 0.111 | 0.115 |
| 0 | 0.108 | 0.158 | 0.160 |
| 5 | 0.108 | 0.155 | 0.159 |
| 10 | 0.108 | 0.153 | 0.159 |
| 15 | 0.109 | 0.151 | 0.158 |
| 20 | 0.108 | 0.150 | 0.159 |
| 25 | 0.108 | 0.150 | 0.157 |
| 30 | 0.108 | 0.149 | 0.157 |

2.8.7 Assessment of Tritosomal Stability of pNP-α-L-Rha (b)

As no activity against α-L-rhamnopyranosides was detected in the previous assay, a higher concentration of pNPαL-Rha (31.0 mg/ml) in DMSO was used in an otherwise identical assay. In addition, the cuvette was incubated at 37° C. for 20 hours and the absorbance reading repeated.

a) Cuvette Setup

| | Blank - volume/μl | Assay - volume/μl |
|---|---|---|
| 10 mM EDTA | 100 | 100 |
| 50 mM GSH | 100 | 100 |
| Substrate solution | 30 | 30 |
| Stock solution | 740 | 740 |
| Tritosomes | 0 | 30 |
| DMSO | 30 | 0 |

b) Readings from Spectrometer

| Time/min | Blank | Assay 1 | Assay 2 |
|---|---|---|---|
| Before addition | 0.126 | 0.128 | 0.124 |
| 0 | 0.130 | 0.173 | 0.172 |
| 5 | 0.127 | 0.169 | 0.170 |
| 10 | 0.127 | 0.166 | 0.167 |
| 15 | 0.128 | 0.165 | 0.166 |
| 20 | 0.128 | 0.162 | 0.164 |
| 25 | 0.128 | 0.161 | 0.165 |
| 30 | 0.127 | 0.158 | 0.163 |
| 20 hours | 0.127 | 0.138 | 0.131 |

2.8.8 Assessment of Tritosomal Stability of pNP-β-D-Gal

A stock solution of p-nitrophenyl β-D-galactopyranoside in DMSO (31.0 mg/ml) was used in an assay equivalent to 2.8.7. In addition, the cuvette was incubated at 37° C. for 20 hours and the absorbance reading repeated.

a) Cuvette Setup

| | Blank - volume/μl | Assay - volume/μl |
|---|---|---|
| 10 mM EDTA | 100 | 100 |
| 50 mM GSH | 100 | 100 |
| Substrate solution | 30 | 30 |
| Stock solution | 740 | 740 |
| Tritosomes | 0 | 30 |
| DMSO | 30 | 0 |

b) Readings from Spectrometer

| Time/min | Blank | Assay 1 | Assay 2 |
|---|---|---|---|
| Before addition | 0.125 | 0.120 | 0.130 |
| 0 | 0.126 | 0.170 | 0.173 |
| 5 | 0.128 | 0.170 | 0.173 |
| 10 | 0.126 | 0.171 | 0.174 |
| 15 | 0.127 | 0.172 | 0.175 |
| 20 | 0.127 | 0.174 | 0.175 |
| 25 | 0.126 | 0.177 | 0.178 |
| 30 | 0.127 | 0.180 | 0.179 |
| 20 hours | 0.124 | 0.412 | 0.402 |

2.8.9 Assessment of Tritosomal Stability of pNP-β-D-Glc

A stock solution of p-nitrophenyl β-D-glucopyranoside in DMSO (31.0 mg/ml) was used in an assay equivalent to 2.8.7. In addition, the cuvette was incubated at 37° C. for 20 hours and the absorbance reading repeated.

a) Cuvette Setup

| | Blank - volume/μl | Assay - volume/μl |
|---|---|---|
| 10 mM EDTA | 100 | 100 |
| 50 mM GSH | 100 | 100 |
| Substrate solution | 30 | 30 |
| Stock solution | 740 | 740 |
| Tritosomes | 0 | 30 |
| DMSO | 30 | 0 |

B) Readings from Spectrometer

| Time/min | Blank | Assay 1 | Assay 2 |
|---|---|---|---|
| Before addition | 0.120 | 0.124 | 0.122 |
| 0 | 0.124 | 0.174 | 0.173 |
| 5 | 0.124 | 0.171 | 0.172 |
| 10 | 0.125 | 0.169 | 0.171 |
| 15 | 0.125 | 0.168 | 0.171 |
| 20 | 0.124 | 0.168 | 0.170 |
| 25 | 0.125 | 0.168 | 0.168 |
| 30 | 0.126 | 0.168 | 0.166 |
| 20 hours | 0.151 | 0.331 | 0.334 |

2.8.10 Determining Extinction Coefficient of pNP in pH5.5 Citrate-Phosphate Buffer

A stock solution of p-nitrophenol (0.2M) in citrate-phosphate buffer (0.2M, pH5.5) was diluted to a series (0.2, 0.1, 0.075, 0.05, 0.025, 0.01, 0.005, 0.0 mM) and incubated in a multiwell plate (200 μl) at 37° C. The absorbance at 405 nm was recorded, with and without pathcheck.

2.8.11 Determining Standard Enzyme Kinetics of N-WT in pH5.5 Citrate-Phosphate Buffer

A solution of p-nitrophenyl α-L-rhamnopyranoside 3.5 mM) in citrate phosphate buffer (0.2M, pH5.5, with and without 0.2% Triton X100) was prepared and diluted into a gradient series (3.5, 3.0, 2.5, 2.0, 1.5, 1.0, 0.5, 0.25 mM). A solution of N-WT (5 mg/ml) in citrate phosphate buffer (0.2M, pH5.5) was prepared. 190 μl substrate solution and 10 μl enzyme solution were incubated at 37° C. with 5+1 second shaking and absorbance at 405 nm read every 6 seconds for 5 minutes.

2.8.12 Assessment of Tritosomal Stability of N-WT

N-WT (5 mg) was dissolved in citrate phosphate buffer (680 μl, 0.2M, pH5.5 plus 1 mM EDTA and 5 mM GSH) and mixed with Triton X100 solution (20 μl, 10%). Tritosome preparation (300 μl) was incubated at 37° C. for 5 minutes and added to the N-WT solution. Aliquots (50 μl) were removed (0, 10, 20, 30, 45, 60, 90 minutes, 2, 3, 4, 6, 12, 24, 48 hours) and diluted to a total of 500 μl in citrate phosphate buffer and a standard kinetic assay carried out.
ⁱ P. R. Crocker, T. Feizi; Curr. Opin. Struc. Biol., 1996, 6, 679-691
ⁱⁱ R. Pulido, F. Sanchezmadrid; European J. Immunology, 1992, 22, 463-468
ⁱⁱⁱ P. M. Rudd, R. A. Dwek; Crit. Rev. Biochem. Mol. Biol., 1997, 32, 1-100
^{iv} W. M. Macindoe et al; Chem. Commun., 1998, 847-848
^{v} I. Niculescu-Duvaz, C. J. Springer; Adv. Drug. Del. Rev., 1997, 26, 151-172
^{vi} P. D. Senter, P. M. Wallace, H. P. Svensson, V. M. Vrudhula, D. E. Kerr, I. Hellström; Bioconjugate Chem., 1993, 4, 3-9
^{vii} P. D. Senter; FASEB J., 1990, 4, 188-193
^{viii} S. K. Sharma; Adv. Drug. Del. Rev., 1996, 22, 369-376
^{ix} S. H. Barondes; Ann. Rev. Biochem., 1981, 50, 207-231
^{x} For example: N. Sharon, H. Lis; Trends. Biochem. Sci., 1987, 12, 488-491
^{xi}G. Ashwell, J. Harford; Ann. Rev. Biochem., 1982, 51, 531-554
^{xii} P. D. Stahl, J. S. Rodman, M. J. Miller, P. H. Schlesinger; Proc. Natl. Acad. Sci, 1978, 75, 1399-1403
^{xiii} V. L. Shepherd, Y. C. Lee, P. H. Schlesinger, P. D. Stahl; J. Biol. Chem., 1984, 259, 2257-2261
^{xiv} A. C. Roche, P. Midoux, P. Bouchard, M. Monsigny; FEBS Letters, 1985, 193, 63-68
^{xv} M. Mutter, G. Beldman, H. A. Schols, A. G. J. Voragen; Plant Physiol., 1994, 106, 241-250
^{xvi} G. Vic, J. J. Hastings, O. W. Haworth, D. H. G. Crout; Tet. Asymm., 1996, 7, (3), 709
^{xvii} J. O. Andrianaivoravelona, C. Terreaux, S. Sahpaz, J. Rasolondrarnanitra, K. Hostettmann; Phytochemistry, 1999, 52, 1145-1148
^{xviii} M. M. Abou-Zaid, C. Nozzolilli; Phytochemistry, 1999, 52, 1629-1631
^{xix} ENZYME online database; http://www.expasy.ch/enzyme/
^{xx} O. Westphal, H. Feier; Chem. Ber., 1956, 582-588
^{xxi} The inventor is grateful to Prof Ruth Duncan and Miss Samantha Kneller, Cardiff University, for a donation of rat liver tritosome preparation.
^{xxii} IODO-GEN technique as described in previous reports
^{xxiii} H. Boyd, S. J. Leach, B. Milligan, Int. J. Peptide Protein Res., 1972, 4, 117-122; Communication from Amersham Biosciences UK
^{xxiv} A. Trouet, Methods Enzymol, 1974, 31, 323
^{xxv} P. K. Smith, R. I. Krohn, G. T. Hermanson, A. K. Mallia, F. H. Gartner, M. D. Provenzano, E. K. Fujimoto, N. M. Goeke, B. J. Olson, D. C. Klenk, Anal. Biochem., 1985, 150, 76-85

1-20. (canceled)

21. A conjugate comprising a rhamnopyranosidase enzyme glycosylated with mannose, galactose, glucose, fucose, N-acetylglucosamine, rhamnose or a combination thereof.

22-28. (canceled)

29. A method for synthesizing a conjugate as claimed claim 21, which method comprises deglycosylating a naturally occurring or synthetic rhamnopyranosidase enzyme and then reglycosylating the enzyme with a lectin-binding group.

30. A method for synthesizing a prodrug comprising a drug moiety and a carbohydrate cap, which comprises contacting the drug in reactive form with a carbohydrate cap donor in the presence of an enzyme.

31-39. (canceled)

40. A method for directing a drug to a cell of a human or animal having a lectin on the surface thereof, which method comprises administering to the human or animal (i) a glycoconjugate comprising an enzyme conjugated to a carbohydrate moiety simultaneously or sequentially with (ii) a prodrug, wherein the glycoconjugate binds to the lectin by the carbohydrate moiety, is endocytosed into the cell, and activates the prodrug by removal of a carbohydrate cap.

41. A method according to claim 40, wherein the carbohydrate moiety is selected from the group consisting of mannose, galactose, glucose, fucose, N-acetylglucosamine and rhamnose.

42. A method according to claim 40, wherein the carbohydrate moiety is mannose.

43. A method according to claim 40, wherein the carbohydrate moiety is galactose.

44. A method according to claim 40, wherein the glycoconjugate comprises from 1 to 20 sugar residues per site at from 1 to 20 sites.

45. A method according to claim 40, wherein the enzyme is an α-L-rhamnopyranosidase.

46. A method according to claim 40, wherein the enzyme is naringinase.

47. A method according to claim 40, wherein the carbohydrate moiety is attached to the enzyme through a lysine or cysteine residue.

48. A method according to claim 40, wherein the carbohydrate moiety is attached to the enzyme through a linker.

49. A method according to claim 40, wherein the carbohydrate moiety is attached to the enzyme through a linker selected from the group consisting of:
(a) an imino alkyl group of the formula Ia
-(c)-SCH₂C(NH)-(e)- Ia
or of the formula Ia′
[-(c)-XCH₂COX(CH₂)n]₂N(CH₂)nNHCO(CH₂)nSCH₂C(NH)-(e)- Ia′
wherein X is N;
(b) a direct link of formula Ib
-(c)-(e)-; Ib
(c) a group of formula Ic
-(c)-(CH₂)nC(O)-(e)- or -(c)C(O)(CH₂)n-(e)-; and Ic
(d) a group of formula Id
-(c)-CONH(CH₂)nNH—C(C(O))═C(C(O))-(e)-, Id
wherein —C(C(O))═C(C(O))— is the 4-membered cyclic squarate; and
wherein -(c)- indicates linkage to the carbohydrate moiety, -(e)- indicates linkage to the enzyme, and each n is independently selected from 1 to 10.

50. A method according to claim 40, wherein the carbohydrate moiety (c) and the enzyme (e) comprise a divalent dendrimeric system of general formula II 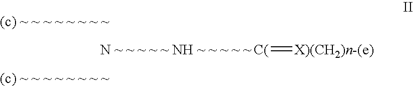
wherein [symbol] indicates a hydrocarbyl moiety, X is O, S, CH₂ or NH and n is from 1 to 10.

51. A method according to claim 40, wherein the carbohydrate moiety (c) and the enzyme (e) comprise a divalent dendrimeric system of general formula II′
[[(c)-C(═X)Y(CH₂)n]v′N(H)v(CH₂)n]v″N(H)ᵥCO(CH₂)nY(CH₂)nC(═X)(CH₂)n-(e) II
wherein each X is independently selected from the group consisting of O, S, CH₂ and NH; each Y is independently selected from the group consisting of NH, S and O; n is from 1 to 10; and the sum of v and v′ and of v and v″ independently equals the valence of N, wherein v is 0 or 1 and v′ and v″ are independently 1 or 2.

52. A method according to claim 40, wherein the glycoconjugate is of the formula IIIb
Rv′N(H)vCO(CH₂)nY(CH₂)nC(═NM)N-(e) IIIb
wherein R comprises a carbohydrate, (e) is the enzyme, v and v′ are each 1, each n is from 1 to 10, and Y is S.

53. A method according to claim 40, wherein the glycoconjugate is selected from the group consisting of wild type naringinase modified by IME-thiogalactoside reagent, wild type naringinase modified by IME-thiomannoside reagent, wild type naringinase modified by IME-dendrithiogalactoside reagent, deglycosylated naringinase, reglycosylated using IME-thiomannoside reagent, wild type naringinase modified by IME-thioglycoside reagent, wild type naringinase modified by IME-dendrithioglycoside reagent, deglycosylated naringinase, reglycosylated using IME-thioglycoside reagent, and deglycosylated naringinase reglycosylated using IME-dendrithioglycoside reagent;
wherein IME is 2-imino-2-methoxyethyl.

54. A method according to claim 40, wherein the prodrug is a rhamnoside of an amine, alcohol or thiol.

55. A method according to claim 40, wherein the lectin is selected from the group consisting of a galactose-specific lectin on hepatocytes, a mannose-specific lectin on macrophages, a mannose- and mannose-6-phosphate-specific lectin on macrophages, and a mannose-6-phosphate-specific lectin on monocytes.

56. A method for directing a drug to a cell of a human or animal having a lectin on the surface thereof according to claim 40, which method comprises administering to the human or animal (i) a glycoconjugate comprising a rhamnopyranosidase enzyme conjugated to a carbohydrate moiety simultaneously or sequentially with (ii) a prodrug, wherein the glycoconjugate binds to the lectin by the carbohydrate moiety and activates the prodrug by removal of a carbohydrate cap.
